(19) United States
(12) Patent Application Publication
(10) Pub. No.: US 2026/0294972 A1
(43) Pub. Date: Oct. 1, 2026

(54) Formulations and Methods for Collecting and Storing Blood and Blood Products

(71) Applicant: Hemerus Medical, Inc., St. Paul, MN (US)

(72) Inventors: Majid Zia, St. Paul, MN (US); John R. Hess, Seattle, WA (US); Nancy C. Wilker, Lexington, MA (US)

(73) Assignee: Hemerus Medical, Inc., St. Paul, MN (US)

(21) Appl. No.: 19/168,635

(22) PCT Filed: Mar. 28, 2024
(86) PCT No.: PCT/US2024/022068

§ 371 Date: Sep. 24, 2025

Related U.S. Application Data
(60) Provisional application No. 63/456,361, filed on Mar. 31, 2023.
(60) Provisional application No. 63/522,708, filed on Jun. 22, 2023.

(51) Int. Cl. A61K 35/18; A61J 1/10; A61K 47/02; A61K 47/12; A61K 47/26

(52) U.S. Cl. CPC A61K 35/18; A61J 1/10; A61K 47/02; A61K 47/12; A61K 47/26

(57) ABSTRACT

In various aspects, the invention provides dry (or solid) compositions or formulations (in the form of powder, capsules, and tablets) to be mixed with donated whole blood. In another aspect, the invention provides dry or solid compositions or formulations (in the form of powder, capsules, and tablets) to be mixed with a donated blood product such as red blood cells. In further aspects, the invention provides a blood bag comprising an interior containing dry (or solid) compositions or formulations (in the form of powder, capsules, and tablets) to be mixed with donated whole blood or a blood product thereof. In further aspects, the invention provides whole blood, or a blood product thereof, mixed with a dry formulation as described herein.

### CROSS REFERENCE TO RELATED APPLICATIONS

This application claims priority benefit from U.S. Provisional Application No. 63/456,361 filed Mar. 31, 2023, and from U.S. Provisional Application No. 63/522,708 filed Jun. 22, 2023, each of which is incorporated by reference herein in its entirety.

### BACKGROUND

The invention relates to the field of blood and blood product collection, processing, and storage.

The collection of whole blood or blood components, such as plasma, red blood cells, or platelets, from blood donors plays a vital role in patient healthcare. While there are currently products and methods for collecting and storing this valuable resource, there is a need for improved products to collect and store donated blood and blood products.

### SUMMARY

In some embodiments, the invention provides improved formulations and bags for collecting and storing blood and blood products for future transfusion to recipient patients.

Accordingly, in a first aspect, the invention provides a dry formulation lacking free H2O molecules, where mixing of said formulation with donated whole blood or a blood product thereof (e.g., red blood cells, platelets, and/or plasma) creates a formulation-treated blood for transfusion into a recipient patient.

In another aspect, the invention provides formulation-treated blood created by mixing donated whole blood or a blood product thereof with the dry formulation described herein. In some embodiments, the formulation-treated blood is whole blood. In some embodiments, the formulation-treated blood is plasma. In some embodiments, the formulation-treated blood is red blood cells. In some embodiments, the formulation-treated blood is platelets. In some embodiments, the formulation-treated blood is leukoreduced.

In yet another aspect, the invention provides a blood bag comprising an interior, said interior containing the dry formulation describes herein. In some embodiments, the bag is sterilized by a sterilization method that is not steam sterilization.

In various embodiments, the formulation is in a form selected from the group consisting of a tablet, granules, powder, capsule, gel (e.g., lacking free water molecules), and a combination of the foregoing.

In some embodiments, the formulation comprises and anticoagulant and/or comprises an anticoagulant activity. In some embodiments, the formulation comprises a preservative. In some embodiments, the formulation comprises an anticoagulant and a preservative.

In some embodiments, the formulation is mixed with 450 ml (+/−10%) of donated whole blood. In some embodiments, the formulation is mixed with 500 ml (+/−10%) of donated whole blood.

In various embodiments, the formulation holds the formulation-treated blood at a pH of between about 6.8 to about 7.6. In some embodiments, the formulation holds the formulation-treated blood at a pH of between about 7.0 to about 7.4. In some embodiments, the formulation holds the formulation-treated blood at a pH of about 7.2.

In various embodiments, the formulation comprises an anticoagulant component and an energy-providing component. In some embodiments, the anticoagulant component comprises citrate. In some embodiments, the energy-providing component is a sugar such as dextrose.

In further embodiments, the formulation described herein further comprises a buffering component. In some embodiments, the buffering component comprises one or more salts, bases, and/or acids that are safe for transfusion. Suitable salts may include sodium phosphate, sodium bicarbonate, sodium citrate, sodium acetate, potassium phosphate, potassium carbonate, potassium acetate, magnesium citrate, magnesium sulfate, and sodium pyrophosphate. Bases may include sodium hydroxide, potassium hydroxide, and magnesium hydroxide in safe concentrations. Acids may include phosphoric acid, citric acid, and acetic acid.

In further embodiments, the formulation described herein further comprise one or more nucleoside or nucleotide providing agent. Such agents may include, but are not limited to, adenine, guanosine, cytidine, uridine, thymidine, and derivatives thereof.

In further embodiments, the formulation described herein further may comprise one or more amino acid such as, for example, L-carnitine, glycine, glutamine, alanine, proline, arginine, taurine, and cysteine.

In further embodiments, the formulation described herein further may comprise one or more sugars and or sugar alcohols such as, for example, glucose, mannitol, sorbitol, xylitol, fructose, dextrose, ribose, and sucrose.

In further embodiments, the formulation-treated blood is stored for at least two days prior to transfusion into the recipient patient. In further embodiments, the formulation-treated blood is stored for at least three days prior to transfusion into the recipient patient. In further embodiments, the formulation-treated blood is stored for at least seven days prior to transfusion into the recipient patient.

In further embodiments, the formulation-treated blood is stored for at least twenty-eight days, at least thirty-five days, at least forty-two days, at least forty-nine days, or at least fifty-six days, prior to transfusion into the recipient patient. In some embodiments, the formulation-treated blood is stored for no longer than sixty days.

In further embodiments, the formulation-treated blood is stored between about 1° C. and about 6° C. such as, for example, at about 4° C. In further embodiments, the formulation-treated blood is stored between about 20° C. and about 24° C. such as, for example, at about 22° C.

In further embodiments, the formulation-treated blood is stored frozen.

### BRIEF DESCRIPTION OF THE DRAWINGS

FIG. 1 is a schematic depicting a standard blood bag containing liquid anticoagulant.

FIG. 2 is a schematic depicting a two bag blood collection system having two in-line blood bags, where liquid coagulant is contained in the interior of the bag for whole blood and liquid red blood cell additive solution is contained in the interior of the bag for packed red blood cells.

FIG. 3 is a schematic depicting a standard blood bag containing a non-limiting formulation of the invention in dry (or solid) formulation form (e.g., powder, capsule, granules, and/or tablet form), not liquid form, for immediate or sustained release of one more of the ingredients.

FIG. 4 is a schematic depicting a standard blood bag containing a non-limiting formulation of the invention in single tablet form.

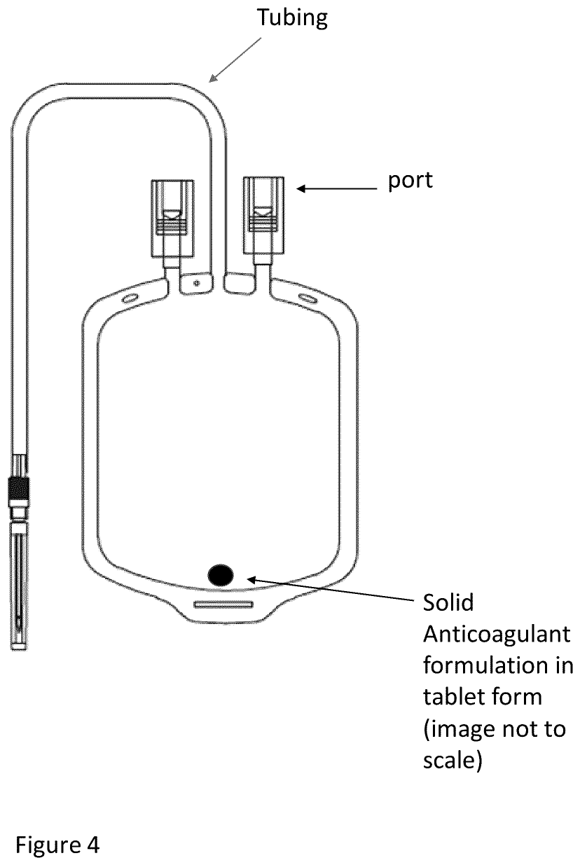

FIG. 5 is a schematic depicting a standard blood bag containing a non-limiting formulation of the invention in multiple tablet form.

FIG. 6 is a schematic depicting a non-limiting embodiment of the invention, namely a multi-layer tablet comprising an anticoagulant, such as a citrate-providing agent, a phosphate-providing agent, and an energy-providing, such as dextrose. As shown in FIG. 6, the sugar layer is sandwiched between the citrate-providing agent layer and the phosphate-providing agent layer.

FIG. 7 is a schematic depicting a non-limiting embodiment of the invention, namely a multi-layer tablet comprising a nucleo-providing agent (e.g., guanosine or adenine), a phosphate-providing agent (e.g., monobasic sodium phosphate monohydrate or sodium phosphate dibasic anhydrous) and an energy-providing (e.g., dextrose) coated with a layer of an anticoagulant, such as a citrate-providing agent.

### DETAILED DESCRIPTION

The invention stems, in part, from the development of blood bags and methods for making and using them that allow for the collection and storage of whole blood as well as blood components including red blood cells, plasma, platelets, and specific components therein, such as cryo-precipitate, immunoglobulins or albumin.

The published patents, patent applications, websites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Terms defined or used in the description and the claims shall have the meanings indicated, unless context otherwise requires. Technical and scientific terms used herein have the meaning commonly understood by one of skill in the art to which the present invention pertains, unless otherwise defined. Any conflict between an art-understood definition of a word or phrase and a definition of the word or phrase as specifically taught in this specification shall be resolved in favor of the latter. As used herein, the following terms have the meanings indicated. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 30%.

As used herein, by “blood” is meant whole blood and blood component products. Whole blood is simply the fluid that circulates throughout an organism, such as a human. Whole blood includes components including cells (e.g., red blood cells, white blood cells, platelets), as well as noncellular components including proteins (e.g., insulin, immunoglobulins, albumin), fatty acids and carbohydrates (e.g., cholesterol and glucose) suspended in a liquid called plasma.

In some embodiments, the species donating the blood is the same species as the recipient patient. In some embodiments, the donor blood is donated by a human and the recipient patient is a human. In some embodiments, the donor and the recipient share the same blood type. In some embodiments, the recipient has a blood type that is compatible with the blood type of the donor. For example, Rh positive recipients are compatible with donors who are either Rh negative or Rh positive. In another example, AB blood type recipients are compatible with donors who are of blood type A, blood type B, or blood type O. In another example, B blood type recipients are compatible with donors who are of blood type B and blood type O. In another example, A blood type recipients are compatible with donors who are of blood type A and blood type O. In another example, O blood type recipients are compatible with donors who are of blood type O.

After collection from a donor, whole blood can be processed to separate and collect individual components from the blood for transfusion to a recipient patient (e.g., a human patient). For example, plasma, and the non-cellular components suspended in plasma, can be separated from the cellular components of blood. Of course, plasma can also include cellular products, but is typically substantially devoid of red blood cells. Two typical plasma products are platelet-rich plasma (PRP) and platelet-poor plasma (PPP). Likewise, the cellular components can be separated from one another. For example, white blood cells and/or platelets can be separated from red blood cells. Platelets can also be separated from other blood components such as platelet rich plasma (PRP). Each of these blood components, upon separation, can be saved, discarded, or returned to the donor, depending upon the need of the intended recipient of the donated blood. For example, apheresis systems, such as plateletpheresis systems or plasmapheresis systems are commercially available and used to extract desired components (e.g., red blood cells, platelets or plasma) from whole blood from a donor. In some embodiments, the component-reduced blood is returned to the donor by the apheresis system.

Since whole blood collected from a donor will clot ex vivo, donated blood is typically treated with an anticoagulant and preservative solution before the whole blood is stored or separated into blood products such as plasma, platelets, and red blood cells (which blood products may also be stored). Currently, donated blood is collected into blood bags that contain an amount of liquid components, namely anticoagulant and preservative components, that, when mixed with whole blood, will prevent the collected blood from clotting and preservative components intended to maintain the quality of the collected blood and blood constituents through further processing and/or long term storage.

Solutions for blood collection and storage containing anticoagulants and preservatives are well known and include Anticoagulant Citrate Phosphate Dextrose Solution (according to the standards of the United States Pharmacopeial Convention (USP)), Anticoagulant Citrate Phosphate Dextrose Adenine Solution (USP), Anticoagulant Citrate Dextrose Solution A (USP), Anticoagulant Citrate Dextrose Solution B (USP), Anticoagulant Sodium Citrate Solution (USP), Anticoagulant Heparin Solution (USP), Anticoagulant sodium citrate (USP), and Ethylenediaminetetraacetic acid Solution (EDTA). Table 1A provides a non-comprehensive list of typical anticoagulants used in the United States and Europe for blood banking. Currently used liquid anticoagulant and preservative solutions are acidic to prevent caramelization of sugars during steam sterilization and long-term storage of blood bag sets.

TABLE 1A
Common Anticoagulant and Preservative Solutions

| | Amount to which 450 ml (+/−10%) | Amount to which 500 ml (+/−10%) |
|---|---|---|
| Name | collected whole blood is added | collected whole blood is added |
| CPD (citrate | 63 mL solution containing 1.66 | 70 mL solution containing 1.79 |
| phosphate | grams trisodium citrate | grams Dextrose (monohydrate), |
| dextrose) | dihydrate, 1.61 grams dextrose | 1.841 grams Sodium Citrate |
| | monohydrate, 188 milligrams | (dihydrate), 209 milligrams Citric |
| | anhydrous citric acid, and 140 | Acid (anhydrous), and 155.4 |
| | milligrams monobasic sodium | milligrams Monobasic Sodium |
| | phosphate monohydrate in water | Phosphate (monohydrate) in water |
| CPDA-1 | 63 mL solution containing 1.66 | 70 mL solution containing 2.233 |
| (citrate phosphate | grams trisodium citrate | grams Dextrose (monohydrate), |
| dextrose adenine | dihydrate, 2.01 grams dextrose | 1.841 grams Sodium Citrate |
| solution) | monohydrate, 188 milligrams | (dihydrate), 209 milligrams Citric |
| | anhydrous citric acid, 140 | Acid (anhydrous), 19.3 milligrams |
| | milligrams monobasic sodium | adenine, and 155.4 milligrams |
| | phosphate monohydrate, and | Monobasic Sodium Phosphate |
| | 17.3 milligrams adenine in water | (monohydrate) in water |
| CP2D (citrate | 63 mL solution containing 3.222 | 70 mL solution containing 3.57 |
| phosphate double | grams dextrose (monohydrate), | grams dextrose (monohydrate), |
| dextrose) | 140 milligrams monobasic | 1.84 grams sodium citrate |
| | sodium phosphate | (dihydrate), 0.229 grams citric acid |
| | monohydrate, 1.66 grams | (monohydrate), and 0.155 grams |
| | sodium citrate (dihydrate), and | monobasic sodium phosphate |
| | 0.206 grams citric acid | (monohydrate) in water |
| | (monohydrate) in water | |
| ACD-A | 63 mL solution containing 1.548 | 70 mL solution containing 1.72 |
| (anticoagulant | grams Dextrose (monohydrate), | grams Dextrose (monohydrate), |
| citrate dextrose) | 1.386 grams Sodium Citrate | 1.54 grams Sodium Citrate |
| | (dihydrate), and 504 milligrams | (dihydrate), and 560 milligrams |
| | Citric Acid (monohydrate) in water | Citric Acid (monohydrate) in water |

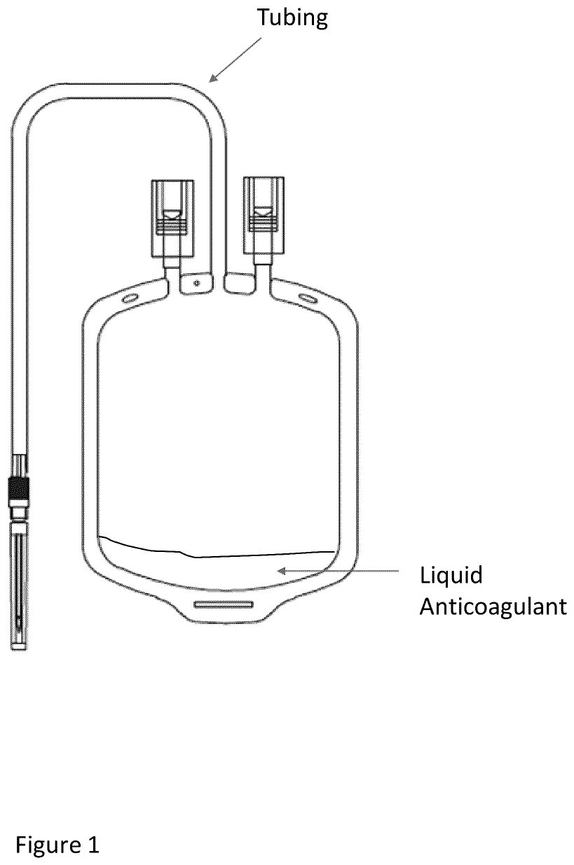

As can be seen from Table 1A, the anticoagulants in use in the United States and other countries (e.g., European and Asian countries) are in liquid form in a blood bag into which collected donor blood is added. FIG. 1 shows a simple blood bag where the liquid anticoagulant is at the bottom of the bag. Donated blood entering the bag will mix, actively or passively, with the liquid anticoagulant using standard blood collection procedures. After the desired volume of donated whole blood (e.g., 450 ml+/−10%, 500 ml+/−10%) is added to the bag while actively or passively mixing, the tubing can be closed off and the bag inverted at least once or repeatedly to thoroughly mix the donor blood with the liquid anticoagulant.

Table 1B provides the formulations of various anticoagulant solutions, where 70 ml of the indicated anticoagulant is added to 500 mL+/−50 ml of collected whole blood from a donor. Typical ratio of anticoagulant to nominal whole blood collection volume is about 0.14.

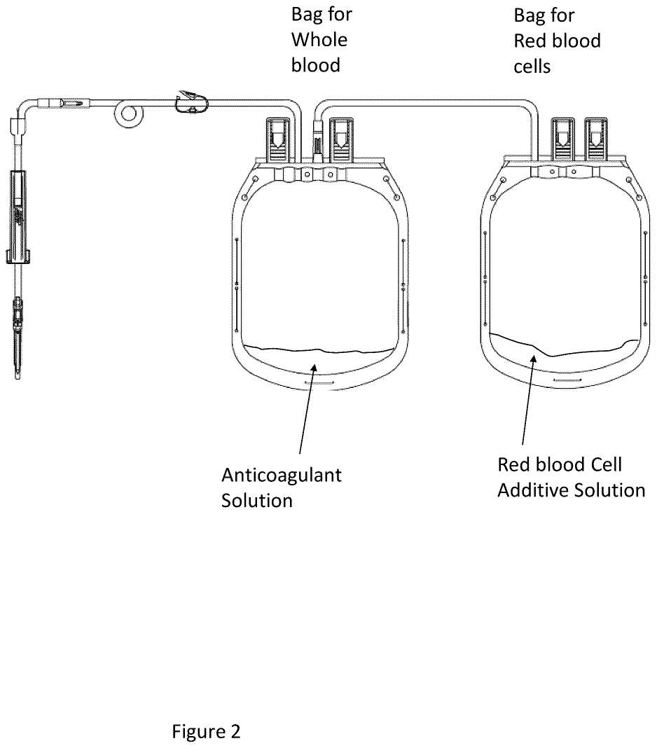

After the donated whole blood is collected and mixed with the liquid anticoagulant, whole blood may be stored for future use and/or separated into desired blood components in an in-line system or dockable system whereby all the contents of the system remain sterile. An in-line system is a system where the blood collection bag containing anticoagulant is directly connected to the donor's needle via tubing, allowing blood to flow directly from the donor into the collection bag. This setup often includes additional bags connected via tubing for the separation and storage of different blood components (e.g., red cells, plasma, platelets) after donation. The in-line system is designed to enhance safety by reducing the risk of contamination and ensuring the integrity of the blood and its components during collection and processing. A dockable system, on the other hand, is a type of blood collection setup where individual components of the blood collection or processing kit are not initially fully integrated and connected. Instead, various components such as the blood collection bag, donor needle, and any additional satellite bags for component separation are separate and can be “docked” or connected together as needed for the blood collection and processing. This docking might involve manual connection of the bags and tubing at specific points in the procedure, which can offer flexibility in customizing the collection kit for different types of donations (e.g., whole blood, apheresis) or in response to specific donor or procedural requirements. In some embodiments, the collected whole blood (i.e., collected from a donor) is collected in such a way that the fluid (e.g., whole blood or a blood product separated therefrom) can transfer from one bag or tube to another in the system without being exposed to external air, thus allowing the fluid in the system to remain sterile. FIG. 2 shows a non-limiting in-line system comprising a first bag for whole blood containing liquid anticoagulant and a second in-line bag containing a liquid red blood cell additive solution. Other systems are available which include one or more additional bags to collect plasma, or other components. Some systems include a leukocyte reduction filter to remove white blood cells from whole blood prior to processing the leukocyte-reduced whole blood into blood components. Some systems include a means to reduce pathogens (e.g., filters, pathogen-specific binding agents) to remove pathogens such as bacteria and/or viruses from the blood prior to or during processing it into blood components.

To produce packed red blood cells, the bag containing the donated whole blood mixed with the liquid anticoagulant is spun in a centrifuge, which allows the separation of the plasma from the red blood cells, with the plasma floating on top of the heavier packed red blood cells. The plasma can be expressed and separated from the whole blood (and can be saved in a separate bag), leaving a small amount of plasma in a layer on top of the red blood cells and/or between the red blood cells. A red blood cell additive solution (stored in an in-line bag connected to the bag retaining the red blood cells) is then added to the red blood cell bag, resulting in red blood cells in additive solution, which is also referred to as packed red blood cells or red blood cells in additive solution. From a volume of 450 mL donated whole blood, a packed red blood cell unit will contain about 200 mL red blood cells, 100 ml of the additive solution, and about 30 mL residual plasma and anticoagulant solution. From a volume of 500 mL donated whole blood, a packed red blood cell unit will contain about 220 mL red blood cells, 110 mL of the additive solution, and about 40 mL residual plasma and anticoagulant solution. Of course, the residual content of the plasma and anticoagulant solution could vary significantly based on the donor's hematocrit.

Similar to anticoagulants, red blood cell additive solutions are well known. For example, Table 2A provides a non-comprehensive list of typical red blood cell additive solutions used in the United States and Europe.

TABLE 2A
Common Red Blood Cell Additive Solutions

| | Amount added to packed red | Amount added to packed red |
|---|---|---|
| | blood cells from 450 ml (+/−10%) | blood cells from 500 ml (+/−10%) |
| Name | collected whole blood | collected whole blood |
| AS-1 (Adsol) | 100 mL solution containing 2.2 | 110 mL solution containing 2.42 |
| | grams dextrose monohydrate, | grams dextrose monohydrate, |
| | 900 milligrams sodium chloride, | 990 milligrams sodium chloride, |
| | 750 milligrams mannitol, and 27 | 825 milligrams mannitol, and |
| | milligrams adenine in water. | 29.7 milligrams adenine in water. |
| AS-3 (Nutricel) | 100 mL solution contains 1.1 | 110 mL solution contains 1.21 |
| | grams dextrose (monohydrate); | grams dextrose (monohydrate); |
| | 588 milligrams sodium citrate | 0.647 grams sodium citrate |
| | (dihydrate); 0.451 g sodium | (dihydrate); 0.451 grams sodium |
| | chloride; 276 milligrams | chloride; 0.304 grams |
| | monobasic sodium phosphate | monobasic sodium phosphate |
| | (monohydrate); 42 milligrams | (monohydrate); 0.046 grams |
| | citric acid (monohydrate); and | citric acid (monohydrate); and |
| | 30 milligrams adenine in water | 0.033 grams adenine in water |
| AS-5 (Optisol) | 100 mL solution containing 877 | 110 mL solution contains 965.2 |
| | milligrams sodium chloride, 900 | mg Sodium Chloride, 991 |
| | milligrams dextrose | milligrams Dextrose |
| | monohydrate, 525 milligrams | (monohydrate), 577.7 milligrams |
| | mannitol, and 30 milligrams | Mannitol, and 33.3 milligrams |
| | adenine. | Adenine in water |
| AS-7 (SOLX) | 100 mL solution containing | 110 mL solution containing |
| | 1.585 grams dextrose | 1.744 grams dextrose |
| | monohydrate, 1.00 grams | monohydrate, 1.102 grams |
| | mannitol, 171.2 milligrams | mannitol, 187.2 milligrams |
| | sodium phosphate (dibasic | sodium phosphate (dibasic |
| | anhydrous), 218.2 milligrams | anhydrous), 240 milligrams |
| | sodium bicarbonate, and 26.9 | sodium bicarbonate, and 29.6 |
| | milligrams adenine in water. | milligrams adenine in water. |

Platelets may also be isolated from donated whole blood by standard methods. Two common methods are the Platelet-Rich Plasma (PRP) method and the Buffy Coat method.

Briefly, for the PRP method, donated whole blood is collected and mixed with an anticoagulant and/or preservative. The anticoagulated blood (i.e., whole blood mixed with an anticoagulant and/or preservative) is then lightly centrifuged, which separates the whole blood into three layers: the bottom layer consisting of red blood cells (RBCs), a middle layer known as the “buffy coat” (which contains white blood cells (WBCs) and platelets), and the top layer of platelet-rich plasma (PRP). The platelet-rich plasma layer, which is at the top, is carefully transferred into a new container while not disturbing the other layers. The collected PRP layer is then centrifuged a second time at a higher speed to concentrate the platelets at the bottom of the container, leaving plasma above the platelets. The concentrated platelets are then separated from most of the plasma, resulting in a smaller volume of platelet concentrate, and the concentrated platelets are resuspended in either plasma or a platelet additive solution to maintain their viability and function until transfusion to a recipient patient.

For the buffy coat method, donated whole blood is collected and mixed with an anticoagulant and/or preservative. The anticoagulated blood is then centrifuged to produce three layers-red blood cells at the bottom, a buffy coat layer in the middle containing white blood cells and platelets, and plasma on the top layer. The buffy coat layer, containing white blood cells and platelets, is then carefully extracted to avoid disturbing the other two layers. The buffy coats from multiple donors (e.g., 4-8 units) are pooled together with a small amount of plasma. The pooled buffy coat is centrifuged a second time to separate and concentrate the platelets from other white blood cells to reduce the risk of febrile non-hemolytic transfusion reactions. The concentrated platelets are then resuspended in plasma or a platelet additive solution.

Additive solutions for platelets are also well known. For example, Table 2B provides a list of ingredients of a typical platelet additive solution, namely Isoplate Solution (PAS-F), that is licensed in the United States.

TABLE 2B
Active Ingredients of a Platelet Additive Solution

| Ingredients | | Amount in 100 mL of |
|---|---|---|
| (Chemical Formula) | Molecular Weight | Solution |
| Sodium Chloride | 58.44 | 0.53 grams |
| (NaCl) | | |
| Sodium Acetate | 136.08 | 0.37 grams |
| Trihydrate | | |
| (CH3COONa•3H2O) | | |
| Potassium Chloride | 74.55 | 0.037 grams |
| (KCl) | | |
| Magnesium Chloride | 203.30 | 0.03 grams |
| Hexahydrate | | |
| (MgCl2•6H2O) | | |
| Dibasic Sodium | 177.98 | 0.008 grams |
| Phosphate | | |
| Dihydrate | | |
| (Na2HPO4•2H2O) | | |
| Monobasic | 136.09 | 0.00082 |
| Potassium | | |
| Phosphate NF | | |
| (KH2PO4) | | |
| Sodium Gluconate | 218.14 | 0.5 grams |
| (C6H11NaO7) | | |

*pH may be adjusted with glacial Acetic Acid USP or Sodium Hydroxide NF pH: 7.4 (7.0-7.8).
*Concentration of Electrolytes (mEq/liter): Sodium 141; Potassium 5; Magnesium 3; Chloride 98; Phosphate (HPO = 4) 1 (0.5 mmole P/liter); Acetate (CH3COO—) 27; Gluconate (HOCH2(CHOH)4COO—) 23.

For the platelet additive solution, platelets from donated whole blood are typically stored in a mixture of about 65% platelet additive solution of Table 2B and about 35% plasma. The platelets thus mixed with the platelet additive solution may be stored for up to 5 days at 20-24° C. with continuous agitation (see also Ringwald J, Zimmermann R, and Eckstein R., Transfus Med Rev 20:158-64, 2006). Recently, the platelet additive solution with the above mix ratios has been licensed in the United States for storage of platelets up to 7 days.

In the current system for blood (and blood product) collection and storage, the anticoagulant and preservative blood component additives (e.g., red blood cell and platelet additives) are provided in liquid format. However, the liquid format of the anticoagulant and preservative has a number of drawbacks.

In view of these drawbacks, in some aspects, the invention provides anticoagulant and/or preservative additive that may be provided to donated whole blood or blood products thereof in a dry or solid format. In some embodiments, the invention provides benefits without some of the drawbacks of liquid base formats.

Currently, as noted above, anticoagulant and/or preservative (for whole blood and plasma), platelet additive solution (for platelet units), and red blood cell additive solution (for packed red blood cells) are in liquid form prior to mixing with whole blood, plasma, separated platelets, or separated red blood cells. As a result, preparation of blood bags containing this liquid is cumbersome and costly. To prepare blood bags for use in collected donated blood, liquid anticoagulant and/or preservative solution and, typically, liquid platelet additive solution and liquid red blood cell additive solution, is added to the bag(s) prior to sterilization. The liquid anticoagulant/preservative solution and the platelet and red blood cell additive solutions contain sugar are thus formulated to be acidic to prevent caramelization of sugars during steam sterilization and long-term storage of blood bag sets. For alkaline additives such as the red blood cell additive solution AS-7, the additive is a bicomponent system where the sugar is separated for the remaining ingredients such that during steam sterilization the sugar is not degraded by steam, thereby preventing caramelization of the sugar during the steam sterilization step. The sterilization occurs by steam sterilization under controlled pressure so that the liquid-containing bags do not explode. Following steam sterilization, blood bags are packaged and heat pasteurized or sterilized.

In addition, the preparation and storage of blood bags containing liquid is costly and bulky. Shipping of blood bags containing liquid anticoagulant is costly as well. Since 1 ml of water weighs about a gram, each blood bag containing 63 or 70 mL of liquid anticoagulant is about 63 or 70 grams heavier than blood bags containing the dry (or solid) compositions and formulations described herein. Using the example of a blood bag containing 70 ml of liquid anticoagulant, while each individual blood bag is only 70 grams (or 0.15 pounds) heavier than a blood bag described herein containing a dry (or solid) anticoagulant formulation, shipping and storage of multiple bags containing liquid components becomes arduous. Also, blood bags containing liquid components are more fragile and susceptible to shipment damage, and leakage due to impact and changes in environmental factors such as temperature and pressure during shipping and storage. For example, the liquid solution may freeze due to low temperature storage conditions, condensate due to high temperature and in both cases result in altered composition, concentration, or stability of the ingredients in the solution or physical damage to components of the blood bag sets, or even to the sterile integrity of the blood bag set itself (e.g., if the frozen liquid expanded during storage and punctured a blood bag set component such as a tubing line). It is not uncommon for thousands of blood bags to be shipped, particularly in situations where there is a high occurrence of injury, such as during war or during a pandemic. Yet, 10,000 bags each containing 70 ml of liquid anticoagulant will weigh over 150 pounds more than the blood bags containing the dry (or solid) anticoagulant formulations described herein and more importantly increase the volume wight of shipment(s).

In addition, the whole blood and blood product bags are not able to be completely filled with whole blood (or a blood product), since some of the interior of the bag is already filled with the liquid solution. For example, for donated whole blood of about 500 mL in volume, 70 mL of the bag is taken up by the 70 mL of liquid anticoagulant/preservative (e.g., liquid CPD—see Table 1A). Therefore, in accordance some aspects of the present invention, the whole blood bag containing a dry formulation as described herein may have an interior that is about 12% smaller (i.e., 70 mL divided by 570 mL equals 12.3%) as compared to a whole blood bag containing liquid formulation (e.g., liquid CPD). Likewise, a red blood cell bag containing a dry formulation, a plasma bag containing a dry formulation, and a platelet bag containing a dry formulation may be smaller than, respectively, a red blood cell bag containing a liquid formulation, a plasma bag containing a liquid formulation, and a platelet bag containing a liquid formulation. Thus, in accordance with various embodiments of the invention, the amount of resources needed to produce the blood bags (e.g., whole blood bags, plasma bags, platelet bags, and red blood cell bags) as described herein is reduced as compared to the amount of resources needed to produce blood bags containing liquid anticoagulant/preservative formulations. Thus, in accordance with various aspect of the present invention, the amount of materials (e.g., PVC or non-PVC polymeric materials (e.g., polyolefins and/or polyurethane) and plasticizer (e.g., DEHP or a non-ortho-phthalate plasticizer such as DINCH, TOTM, ATBC, DEHT and/or BTHC).

In yet another advantage of the present invention, given that a standard blood bag for a donated whole blood volume of approximately 570 mL is able to hold an additional 70 mL of liquid formulation, in some embodiments, the amount of whole blood collected into a blood bag is increased. Currently, the maximal amount of whole blood from a single human donor is about 500 mL. But, if a donor (e.g., a human donor) is of an appropriate weight and has an appropriate hematocrit, that donor may be able to donate more than 500 ml of whole blood. In accordance with various aspects of the present invention, that single donor is able to donate 570 mL whole blood to a blood bag containing a dry anticoagulant/preservative formulation as described herein because 70 mL of the volume in the blood bag is not taken up by 70 mL of liquid anticoagulant/preservative formulation. This increased amount of volume of whole blood (i.e., 12.3% more) reduces the amount of resources needed to process the whole blood including, if desired, leukoreducing the blood and/or separating the whole blood into blood products such as plasma, platelets, and red blood cells.

In some embodiments, the present invention is based on the surprising discovery that anticoagulant/preservative in a dry solid form is able to dissolve in and mix with donated whole blood rapidly such that thorough mixing is achieved. By “mixing” is simply meant the dry formulation is combined with the whole blood or blood product. This can be achieved by adding the dry formulation to the whole blood (or blood product), or by adding the whole blood (or blood product) to the dry formation, such as adding whole blood (or blood product (to blood bag already containing a dry formulation as described herein. Also, the amount of some of the constituent chemicals may be reduced as no free water is added. In some embodiments, some ingredients are added to the formulation described herein based on consumption by cells in the whole blood or blood product. In some embodiments, some ingredients are added to the formulation described herein based on maintaining or preventing a concentration gradient across cellular components. In some embodiments, some ingredients are added to the formulation described herein to maintain appropriate pH for storage stability of some components such as the sugars that may caramelize during heat or steam sterilization and long term blood bag storage. The dilution of blood and/or blood products with liquid anticoagulant and/or preservation ingredients may lead to dilution of the coagulation factors and overload blood circularly system in transfused patients. As a result, sterilizing blood bags containing anticoagulant in a dry or solid form is faster and cheaper and likely lead to better and safer outcome in patients. With the lack of fluid, steam sterilization is not required, and so caramelization of components of the anticoagulant is avoided. In addition, the formulation may include acid and bases in the same bag. Note that the amount of acid may also be reduced, and a higher pH may be achieved in a single whole blood container configuration as, unlike blood bag sets with liquid ingredients, with the dry formulations described herein, no dual bag system is needed for the formulation to prevent caramelization. For example, blood bags containing a dry or solid anticoagulant and/or preservative formulation can be sterilized by ionizing radiation such gamma sterilization, x-ray sterilization, e-beam sterilization, ethylene oxide (ETO) sterilization, etc. Note that because only the energy-providing agent (e.g., dextrose) will caramelize during sterilization by dry heat, if the formulation includes separate components (e.g., a tablet of an energy-providing agent and a separate tablet of anticoagulant), blood bags containing the tablet of anticoagulant but not containing the tablet of energy-providing agent can be sterilized by dry heat.

Storage of blood bags containing a dry or solid form of anticoagulant and/or preservative is also easier compared to the storage of blood bags containing a liquid anticoagulant. For example, blood bags containing a dry (or solid) anticoagulant and/or preservative can be folded and/or stacked with high efficiency, reducing the overall volume of the blood bag set. Weight-wise, blood bags containing dry (or solid) anticoagulant are much lighter and occupy less space than blood bags containing liquid anticoagulant.

Another drawback is that by adding the anticoagulant and/or preservative in a liquid format, the donor blood is diluted, thereby diluting all components in blood including cellular and no-cellular components of blood, such as red blood cells and platelets, as well as non-cellular components including clotting factors antibodies, complement proteins, etc. This dilution reduces the activity and therapeutic efficacy of the blood or blood product when it is transfused into a recipient patient. The dilution not only lessens treatment effectiveness but also raises the risk of complications such as transfusion-associated circulatory overload (TACO). In addition, the presence of water in these systems increases the risk of bacterial contamination, shortens the bloods' shelf life, compromises stability, and necessitates higher concentrations of active ingredients in both the blood bags containing the liquid components, but also in the donor blood collected and stored in the liquid component-containing blood bags.

Accordingly, in various aspects, the invention provides compositions and formulations that do not require dilution of whole blood and other blood products (e.g., red blood cells, platelets, and plasma) when added to whole blood to anticoagulated it and/or preserve it. Currently, since collected whole blood is added to a liquid anticoagulant/preservative, the volume of the whole blood increases by about 14% (e.g., 63 mL liquid anticoagulant/preservative added to 450 mL collected whole blood and 70 ml liquid anticoagulant/preservative added to 500 mL collected whole blood). As a result, all components in whole blood (cells and plasma) are diluted. For example, if 500 ml of whole blood is collected into a bag containing 70 mL liquid anticoagulant/preservative, the whole blood is diluted by about 14% (i.e., 70 mL anticoagulant/preservative divided by 500 ml whole blood), and the concentration of cellular components in anticoagulated whole blood (e.g., red blood cells, platelets, white blood cells) per volume of whole blood is about 12.3% lower than that in the originally donated whole blood without the addition of anticoagulant/preservative (i.e., 1 minus (500 mL whole blood divided by 570 mL total anticoagulant/preservative plus whole blood)). Because 500 ml of whole blood is composed of about 220 ml red blood cells and 280 mL plasma (since the average hematocrit of a healthy adult is about 44%), the plasma portion of whole blood (e.g., including antibodies, clotting factors, insulin, blood sugars, etc.) is diluted by about 25% (i.e., 70 mL anticoagulant divided by 280 mL plasma), and the concentration of plasma components are about 20% lower than that in the plasma of the originally donated whole blood without the addition of anticoagulant (i.e., 1 minus (280 mL plasma divided by 350 ml total of anticoagulant/preservative plus plasma)). These dilution factors are significant and would have significant impact on patient care since no matter how much blood is transfused, the normal homeostatic concentrations of blood constituents will not be achieved by transfusing blood or blood products diluted by liquid anticoagulant/preservative.

Likewise, the red blood cell components are also diluted using liquid red blood cell additive. For example, in standard practice using liquid anticoagulant/preservative and liquid red blood cell additive, from 450 mL of donated whole blood, once the majority of the plasma is removed, about 230 ml of red blood cells in anticoagulated plasma (i.e., residual plasma mixed with residual/preservative) remains, to which 100 ml of a liquid red blood cell additive solution is added creating a packed red blood cell unit with a volume of about 330 ml. Likewise, from 500 ml of donated whole blood, once the majority of plasma and leukocytes are removed, about 255 mL of red blood cells in plasma remains, to which 110 mL of red blood cell additive solution is added, creating a packed red blood cell unit of about 367 mL in volume. The mean anticoagulant volumes present in red blood cells in liquid additive solution is about 6.5% of the original anticoagulant volume and the mean amount of plasma in red blood cell in additive solution is about 10% of original plasma in whole blood collected. Adoption of one or more aspects of the present invention will reduce the dilution of whole blood, reduce the dilution of blood products (e.g., packed red blood cells and plasma) and will increase the ease of manufacturing, sterilizing, shipping, and storing blood bags and blood bag systems to blood donor sites as well as to the recipient patients.

Accordingly, in a first aspect, the invention provides a dry (or solid) formulation lacking free H2O molecules, where the mixing of the formulation with whole blood or a blood product thereof (e.g., plasma, platelets, or red blood cells) creates a formulation-treated blood for transfusion into a recipient patient.

By “formulation-treated blood” is meant whole blood or a blood product (e.g., plasma, red blood cells, or platelets) that is mixed with a formulation of the invention that can be stored, either at room temperature, at refrigeration (e.g., 4 degrees Celsius) or frozen, following collection of the blood or blood product and prior to transfusion of the blood or blood product into a recipient patient. In other words, a formulated-treated blood of the invention is beneficial to the recipient patient following storage. A recipient patient is of the same species as that of the donor. For example, if blood is donated by a dog, the recipient patient is a dog. The invention contemplates that other species that can serve as blood donors (and, consequently blood recipients) include, without limitation, pets (e.g., cats, dogs, parrots), livestock animals (e.g., pigs, sheep, cows, ostriches), wool-providing animals (e.g., goats, alpaca, sheep), working animals (e.g., elephants, horses), and exotic and/or endangered animals (e.g., lions, tortoises, blue whales). In some embodiments, the donor (and the recipient patient) is a human. A human recipient patient may be a human of any age or gender in need of a blood transfusion for any reason and includes, without limitation, trauma patients, chemotherapy patients, radiotherapy patients, hemophilia patients, and the like. A formulation-treated blood of the invention is beneficial to the recipient patient by, for example, maintaining clotting activity (e.g., if the formulation-treated blood is plasma or whole blood), or including living cells (e.g., if the formulation-treated blood is red blood cells or plasma).

In some embodiments, the blood product is plasma, platelets, or red blood cells.

In some embodiments, the dry or solid formulation as described herein comprises an anticoagulant ingredient or comprises an anticoagulant activity.

It should be noted that, as used throughout the present description and claims, the words “ingredient” and “component” are used interchangeably.

As used herein, by “anticoagulant” or “anticoagulant ingredient” or having “anticoagulant activity” is meant a formulation as described herein includes a chemical that when combined with whole blood or a blood product (e.g., plasma, platelets, and/or red blood cells) prevents or reduces the coagulation of blood and thus delays or prevents blood clot formation. Non-limiting anticoagulant ingredients include citrate (in the form of, for example, sodium citrate or citric acid), oxalate (in the form of, for example, dimethyl oxalate, oxalic acid, or sodium oxalate), heparin (and derivatives thereof), coumarin and other vitamin K antagonists (e.g., warfarin, brodifacoum, acenocoumarol, phenprocoumon, atromentin, and difenacoum, and phenindione), ethylenediaminetetraacetic acid (commonly referred to as EDTA), direct thrombin inhibitors (e.g., dabigatran) and factor Xa inhibitors (e.g., rivaroxaban, apixaban, betrixaban, and edoxaban).

In some embodiments, the dry or solid formulation as described herein comprises a preservative or comprises a preservative activity.

As used herein, by “preservative”, “storage” or having “preservative activity” is meant that the formulation includes ingredients that, when combined with whole blood or a blood product (e.g., plasma, platelets, and/or red blood cells) preserves the whole blood or blood product during storage. For example, if the blood product includes red blood cells, a formulation comprising a preservative will maintain the red blood cells stored in such a preservative-containing formulation in a healthier state (e.g., more living cells, more ability to uptake oxygen, more ability to maintain shape) as compared to red blood cells stored in a formulation lacking a preservative. Likewise, if the blood product includes platelets, a formulation comprising a preservative will maintain the platelets stored in such a preservative-containing formulation in a healthier state (e.g., more living platelet cells) as compared to red blood cells stored in a formulation lacking a preservative. If the blood (e.g., whole blood) or blood product includes plasma, a formulation comprising a preservative will maintain the components within the whole blood or plasma stored in such a preservative-containing formulation in a healthier state (e.g., more complement activity, more functional immunoglobulin, more functional albumin) as compared to whole blood or a blood product (e.g., plasma) stored in a formulation lacking a preservative. It shall be understood that a combination of ingredients may provide a preservative activity. For example, a nucleo-providing agent (e.g., adenine), sodium (e.g., from sodium citrate or sodium chloride), and an energy-providing component (e.g. dextrose and/or mannitol) may work in concert to preserve stored whole blood cells.

In some embodiments, a formulation-treated blood is stored for at least seven days at room temperature after collection from the donor and before transfusion to the recipient patient. In some embodiments, a formulation-treated blood can be stored for at least five days at room temperature. In some embodiments, a formulation-treated blood can be stored for at least three days at room temperature after collection from the donor and before transfusion to the recipient patient. In some embodiments, a formulation-treated blood can be stored for at least 48 hours at room temperature after collection from the donor and before transfusion to the recipient patient. In some embodiments, a formulation-treated blood can be stored for at least seven days at about 4° C. (or between about 1° C. to about 6° C.). In some embodiments, a formulation-treated blood can be stored for at least five days at about 4° C. (or between about 1° C. to about 6° C.) after collection from the donor and before transfusion to the recipient patient. In some embodiments, a formulation-treated blood can be stored for at least three days at about 4° C. (or between about 1° C. to about 6° C.) after collection from the donor and before transfusion to the recipient patient. In some embodiments, a formulation-treated blood can be stored for at least 48 hours at about 4° C. (or between about 1° C. to about 6° C.) after collection from the donor and before transfusion to the recipient patient. In some embodiments, a formulation-treated blood can be stored frozen for at least seven days after collection from the donor and before transfusion to the recipient patient. In some embodiments, a formulation-treated blood can be stored frozen for at least five days after collection from the donor and before transfusion to the recipient patient. In some embodiments, a formulation-treated blood can be stored frozen for at least three days. In some embodiments, a formulation-treated blood can be stored frozen for at least 48 hours after collection from the donor and before transfusion to the recipient patient.

In some embodiments, the formulation-treated blood is stored (e.g., frozen, at room temperature, or under refrigeration) for at least twenty-eight days, at least thirty-five days, at least forty-two days, at least forty-nine days, or at least fifty-six days, prior to transfusion into the recipient patient. In some embodiments, the formulation-treated blood is stored for no longer than sixty days prior to transfusion into the recipient patient.

In some embodiments, the dry formulations described herein comprise both an anticoagulant and a preservative. Of course, it is known that the same ingredient may act as both an anticoagulant and a preservative. For example, the ingredient sodium citrate may act as both an anticoagulant and as a preservative. A formulation containing both anticoagulant and preservative may be referred to herein as “anticoagulant/preservative formulation”.

In some embodiments, the dry formulations described herein comprise anticoagulant ingredients and do not comprise preservative ingredients.

In some embodiments, the dry formulations described herein comprise preservative ingredients and do not comprise anticoagulant ingredients.

In some embodiments, the invention provides dry (or solid) compositions or formulations (in the form of powder, granules, tablets, and/or capsules) to be mixed with about 500 mL (+/−10%) of donated whole blood. In some embodiments, the dry (or solid) compositions or formulations described herein are to be mixed with about 500 mL (+/−10%) of donated whole blood without the addition of any water. In another aspect, the invention provides dry (or solid) compositions or formulations (in the form of powder, capsules, and/or tablets) to be mixed with a target volume about 450 mL (+/−10%) of donated whole blood. In some embodiments, the dry (or solid) compositions or formulations described herein are to be mixed with typically about 450 mL (+/−10%) of donated whole blood without the addition of any water.

The ordinarily skilled person will, of course, understand that any volume of donated blood can be mixed with the dry (or solid) compositions or formulations described herein. For example, for the CPD anticoagulant/preservative formulation, 1.79 grams of dextrose (monohydrate) is dissolved in 70 mL water and added to 500 mL whole blood collected from a donor. If, for example, only about 400 mL whole blood is collected, the amount of dextrose and the volume it is dissolved in can be adjusted by the whole blood ratio of 400 mL over 500 mL (0.8). Therefore, for 400 mL whole blood collection, the mass of dextrose and the volume of water the dextrose is dissolved in may be calculated as below:

$$Mass of dextrose monohydrate = 400 / 500 \times 1.79 g = 1.43 grams and$$

$$Volume of Water = 400 / 500 \times 70 mL = 56 mL$$

This same calculation can be made for any volume of collected whole blood or blood product from a donor.

Per standard blood banking practice, a 10% deviation in blood volume collection is permissible without any specially labeling change requirements such as “CPD WHOLE BLOOD-LOW VOLUME”. Low volume labeling requirements are needed where the ratio of whole blood collection is between 2/3 and 9/10 of the stated blood volume to be collected with the blood collection set. In addition, per the blood banking standards, volumes lower than ⅔ of the stated volume are not allowed.

In some embodiments, the dry (or solid) compositions and formulations of the invention do not contain any free water molecules. In particular embodiments, the dry (or solid) compositions and formulations of the invention lack free H2O molecules. By a “free H2O molecule” is meant a water molecule that is not bonded, via a covalent or non-covalent bond, to another molecule. In some embodiments, a free H2O molecule as described herein is not ionically bound or covalently bound to another molecule or atom in a given dry or solid formulation as described herein. In some embodiments, a free H2O molecule as described herein is not hydrogen bound to a molecule that is not another H2O molecule. Note that while some of chemical components of the compositions and formulations of the invention may incorporate one or more water molecules, H2O, as part of their chemical structure, the dry (or solid) compositions and formulations of the invention do not comprise any free H2O molecules, that is, the dry formulation does not comprise any molecules of H2O that are not bound by a covalent bond or a non-covalent bond (e.g., ionic bond) to another atom.

In one nonlimiting example, sodium citrate dihydrate, a hydrated form of a chemical used in some of the formulations described herein, has the following chemical structure:

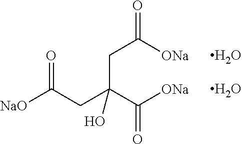

However, as depicted in the above, the two H2O molecules in sodium citrate dihydrate are not free, as each H2O molecule in sodium citrate dihydrate is noncovalently bonded to an —ONa group, where the noncovalent bond is depicted as a *.

In some embodiments, the formulation the formulation comprises an anticoagulant, a preservative, and an energy-providing component.

In some embodiments, the anticoagulant is citrate. By “citrate” is meant a chemical compound that provides citrate ions. A citrate ion has the formula, for example, of C₃H₅O(COO)₃³⁻ which is citric acid minus three H⁺ ions. In the example of sodium citrate dihydrate above, the citrate ions are provided by sodium citrate dihydrate. Additional non-limiting providers of citrate ions are magnesium citrate and citric acid.

In some embodiments, where the formulation includes a preservative, the preservative is also citrate.

In some embodiments, where the formulation includes an energy-providing components, the energy-providing component is a sugar such as dextrose (D-glucose). In some embodiments, the energy-providing component is a monosaccharide, such as glucose, fructose, or galactose.

In some embodiments, the where the formulation includes an energy-providing components, the energy-providing component is a sugar alcohol such as mannitol. In some embodiments, the energy-providing component is a six carbon sugar alcohol such as mannitol, sorbitol, fucitol, inositol, sorbitol, iditol, or galactitol. In some embodiments, the energy-providing component is a five carbon sugar alcohol such as arabitol, xylitol, or ribitol. In some embodiments, the energy-providing component is a four carbon sugar alcohol such as erythritol, or threitol. In some embodiments, the energy-providing component is a seven carbon sugar alcohol such as volemitol. In some embodiments, the energy-providing component is a twelve carbon sugar alcohol such isomalt, malitol, or lactitol.

In various embodiments, the formulation-treated blood has a pH of between about 6.8 to about 7.6. In some embodiments, the formulation-treated blood has a pH of between about 7.0 to about 7.4. In some embodiments, the formulation-treated blood has a pH of about 7.2.

Maintaining the pH of the formulation-treated blood prior to transfusion in the recipient patient may be helpful to maintain a healthier state of components in the formulation-treated blood as compared to components in blood not mixed with the formulation. For example, for red blood cells, healthy red blood cells are recovered when ATP levels are high. Thus, it may be useful to keep the RBC intracellular pH at a level as close to 7.2 as possible without actually exceeding pH 7.2 for extended time during storage. During storage, the adenosine tri phosphate (ATP) concentration characteristically remains level or even increases for a period of time early in storage and then declines. When the RBC ATP concentration falls below 2 μmol/g Hb, RBC recovery is typically below 75%. RBC's lose 2,3-diphosphoglycerate (DPG) early in storage. The rate of synthesis of 2,3-DPG is a function of pH, occurring in excess above pH 7.2 but with breakdown of 2, 3 DPG favored below a pH of 7.2. However, if 2, 3 DPG molecule formation increases, ATP synthesis declines. Therefore, if the pH of stored red blood cells is maintained at close to (but not more than) 7.2, more ATP will be synthesized than 2,3 DPG. Maintenance of pH in whole blood and blood products other than red blood cells (e.g., platelets and plasma) is also important to maintain as much activity as possible in the stored whole blood or blood product prior to transfusion into a recipient patient.

In some embodiments, the formulation further comprises a buffering component. By “buffering component” is meant a molecule (e.g., ion) that aids the formulation in resisting change in pH, for example, if the formulation is mixed with whole blood (or a blood product thereof) and/or stored over time (e.g., longer than two days). In some embodiments, the buffering component is a phosphate, a citrate, an acetate, and/or a carbonate.

Citrate is as defined above.

As used herein, by “acetate” is meant a chemical compound that provides acetate ions. An acetate ion has the formula, for example, of:

C₂H₃O₂⁻

Acetate ions can be provided, for example, by sodium acetate or other acetate-providing molecules.

As used herein, by “phosphate” is meant a chemical compound that provides phosphate or diphosphate ions. A phosphate ion has the formula, for example, of: PO₄³⁻. A diphosphate ion has the formula, for example, of P₂O₇⁴⁻. Phosphate ions can be provided, for example, by sodium phosphate (e.g., monobasic sodium phosphate or dibasic sodium phosphate), or other phosphate-providing molecules. Diphosphate ions can be provided, for example, by diphosphate salt (e.g., tetrasodium pyrophosphate), or other diphosphate-providing molecules.

As used herein, by “carbonate” is meant a meant a chemical compound that provides carbonate or bicarbonate ions. A carbonate ion has the formula, for example, of: CO₃²⁻. Carbonate ions can be provided, for example, by sodium hydrogen carbonate and other carbonate-providing molecules. A bicarbonate ion has the formula, for example, of HCO₃⁻. Bicarbonate ions can be provided, for example, by sodium bicarbonate, potassium bicarbonate, magnesium bicarbonate and/or other bicarbonate-providing molecules.

Tables 3A, 3B, 4A and 4B below provide non-limiting lists of chemical components of various anticoagulant/preservative formulations that can, in various embodiments of the invention, be added, in dry or solid composition form, to 500 mL (+/−10%) of donated whole blood without the addition of water. The amount of hydration of each ingredient may be different and may be calculated by those skilled in the art. In some embodiments, Tables 3A and 3B represent examples of the lower limit of chemicals to be added to 500 ml of donated whole blood, and in another and/or additional example, in some embodiments, Tables 4A and 4B represent examples of the upper limit of chemicals to be added to 500 ml of donated whole blood.

TABLE 3A
Composition of non-limiting dry anticoagulant/preservative formulations
to be combined with 500 mL of donated whole blood (lower limit).

| | | Chemical | | | | |
|---|---|---|---|---|---|---|
| Chemical | Chemical | MW | ACDA | CPD | CP2D | CPDA-1 |
| Name | Formula | (g/mol) | (mg) | (mg) | (mg) | (mg) |
| Sodium | C6H5Na3O7 | 258.06 | 1040.16 | 1243.46 | 1243.46 | 1243.46 |
| Citrate | | | | | | |
| Citric Acid | C6H8O7 | 192.124 | 410.61 | 167.84 | 167.84 | 167.84 |
| Monobasic | NaH2PO4 | 119.98 | — | 103.05 | 103.05 | 103.05 |
| Sodium | | | | | | |
| Phosphate | | | | | | |
| Dextrose | C6H12O6 | 180.16 | 1246.78 | 1297.67 | 2595.34 | 1618.83 |
| Adenine | C5H5N5 | 135.13 | — | — | — | 16.89 |

| | | | | |
|---|---|---|---|---|
| Total Weight of chemicals for 500 mL | 2697.6 | 2812.0 | 4109.7 | 3150.1 |
| whole blood collection (mg) | | | | |

TABLE 3B
Composition of non-limiting dry anticoagulant/preservative formulations
to be combined with 500 mL of donated whole blood (still lower limit).

| Chemical | Chemical | MW | ACDA | CPD | CP2D | CPDA-1 |
|---|---|---|---|---|---|---|
| Name | Formula | (g/mol) | (mg) | (mg) | (mg) | (mg) |
| Sodium | C6H5Na3O7 | 258.06 | 1083 | 1225 | 1225 | 1225 |
| Citrate | | | | | | |
| Citric Acid | C6H8O7 | 192.124 | 409 | 157 | 157 | 157 |
| Monobasic | NaH2PO4 | 119.98 | — | 101 | 101 | 101 |
| Sodium | | | | | | |
| Phosphate | | | | | | |
| Dextrose | C6H12O6 | 180.16 | 1250 | 1232 | 2466 | 1542 |
| Adenine | C5H5N5 | 135.13 | — | — | — | 13 |

| | | | | |
|---|---|---|---|---|
| Total Weight of chemicals for 500 mL | 2742 | 2715 | 3949 | 3038 |
| whole blood collection (mg) | | | | |

In the embodiment set forth in Tables 3A and 3B above, the non-limiting example of the lower limit is defined as 450/513 ratio of nominal, or 12.3% below nominal. In another embodiment, Table 3B shows a non-limiting example of a change of 5% below 280/(280+70) or 0.80 of nominal or 25% below nominal.

TABLE 4A
Composition of non-limiting dry anticoagulant/preservative formulations
to be combined with 500 mL of donated whole blood (upper limit).

| | | Chemical | | | | |
|---|---|---|---|---|---|---|
| Chemical | Chemical | MW | ACDA | CPD | CP2D | CPDA-1 |
| Name | Formula | (g/mol) | (mg) | (mg) | (mg) | (mg) |
| Sodium | C6H5Na3O7 | 258.06 | 1245.07 | 1488.42 | 1488.42 | 1488.42 |
| Citrate | | | | | | |
| Citric Acid | C6H8O7 | 192.124 | 491.50 | 200.90 | 200.90 | 200.90 |
| Monobasic | NaH2PO4 | 119.98 | — | 123.35 | 123.35 | 123.35 |
| Sodium | | | | | | |
| Phosphate | | | | | | |
| Dextrose | C6H12O6 | 180.15804 | 1492.40 | 1553.31 | 3106.63 | 1937.74 |
| Adenine | C5H5N5 | 135.12985 | — | — | — | 20.21 |

| | | | | |
|---|---|---|---|---|
| Total Weight of chemicals for 500 mL | 3229.0 | 3366.0 | 4919.3 | 3770.6 |
| whole blood collection (mg) | | | | |

TABLE 4B
Composition of non-limiting dry anticoagulant/preservative formulations
to be combined with 500 mL of donated whole blood (still upper limit).

| Chemical | Chemical | MW | ACDA | CPD | CP2D | CPDA-1 |
|---|---|---|---|---|---|---|
| Name | Formula | (g/mol) | (mg) | (mg) | (mg) | (mg) |
| Sodium | C6H5Na3O7 | 258.06 | 1523 | 1699 | 1699 | 1699 |
| Citrate | | | | | | |
| Citric Acid | C6H8O7 | 192.124 | 577 | 221 | 221 | 221 |
| Monobasic | NaH2PO4 | 119.98 | — | 143 | 143 | 143 |
| Sodium | | | | | | |
| Phosphate | | | | | | |
| Dextrose | C6H12O6 | 180.16 | 1755 | 1705 | 3409 | 2134 |
| Adenine | C5H5N5 | 135.13 | — | — | — | 21 |

| | | | | |
|---|---|---|---|---|
| Total Weight of chemicals for 500 mL | 3855 | 3768 | 5472 | 4218 |
| whole blood collection (mg) | | | | |

In the non-limiting embodiment set forth in Table 4A above, the upper limit is defined as 1.05× nominal. In the still upper limit in the non-limiting example in Table 4B, this upper limit is changed to 5% above nominal.

Since some blood collection centers also collect 450 ml of donated whole blood, in yet another embodiment, Tables 5A, 5B, 6A, and 6B below provide non-limiting lists of chemical components of various dry anticoagulant/preservative formulations that can be added, in solid compound form, to 450 mL (+/−10%) of donated whole blood without the addition of water, where. In yet additional examples, Tables 5A and 5B represents non-limiting examples of the lower limit of chemicals to be added to 450 ml of donated whole blood, and Tables 6A and 6B represents non-limiting examples of the upper limit of chemicals to be added to 450 mL of donated whole blood.

TABLE 5A
Composition of non-limiting dry anticoagulant/preservative formulations
to be combined with 450 mL of donated whole blood (lower limit)

| | | Chemical | | | | |
|---|---|---|---|---|---|---|
| Chemical | Chemical | MW | ACDA | CPD | CP2D | CPDA-1 |
| Name | Formula | (g/mol) | (mg) | (mg) | (mg) | (mg) |
| Sodium Citrate | C6H5Na3O7 | 258.06 | 936.14 | 1119.12 | 1119.12 | 1119.12 |
| Citric Acid | C6H8O7 | 192.124 | 369.55 | 151.05 | 151.05 | 151.05 |
| Monobasic | NaH2PO4 | 119.98 | — | 92.74 | 92.74 | 92.74 |
| Sodium | | | | | | |
| Phosphate | | | | | | |
| Dextrose | C6H12O6 | 180.16 | 1122.10 | 1167.90 | 2335.81 | 1456.94 |
| Adenine | C5H5N5 | 135.13 | — | — | — | 15.20 |

| | | | | |
|---|---|---|---|---|
| Total Weight of chemicals for 450 mL | 2427.8 | 2530.8 | 3698.7 | 2835.1 |
| whole blood collection (mg) | | | | |

TABLE 5B
Composition of non-limiting dry anticoagulant/preservative
formulations to be combined with 450 mL of donated
whole blood (still lower limit)

| Chemical | Chemical | MW | ACDA | CPD | CP2D | CPDA-1 |
|---|---|---|---|---|---|---|
| Name | Formula | (g/mol) | (mg) | (mg) | (mg) | (mg) |
| Sodium | C6H5Na3O7 | 258.06 | 995 | 1127 | 1127 | 1127 |
| Citrate | | | | | | |
| Citric Acid | C6H8O7 | 192.124 | 377 | 146 | 146 | 146 |
| Monobasic | NaH2PO4 | 119.98 | — | 94 | 94 | 94 |
| Sodium | | | | | | |
| Phosphate | | | | | | |
| Dextrose | C6H12O6 | 180.16 | 1149 | 1132 | 2265 | 1416 |
| Adenine | C5H5N5 | 135.13 | — | — | — | 13 |

| | | | | |
|---|---|---|---|---|
| Total Weight of chemicals | 2521 | 2499 | 3632 | 2796 |
| for 450 mL whole | | | | |
| blood collection (mg) | | | | |

TABLE 6A
Composition of non-limiting dry anticoagulant/preservative
formulations to be combined with 450 mL of donated
whole blood (upper limit)

| | | Chemical | | | | |
|---|---|---|---|---|---|---|
| Chemical | Chemical | MW | ACDA | CPD | CP2D | CPDA-1 |
| Name | Formula | (g/mol) | (mg) | (mg) | (mg) | (mg) |
| Sodium | C6H5Na3O7 | 258.06 | 1120.56 | 1339.58 | 1339.58 | 1339.58 |
| Citrate | | | | | | |
| Citric Acid | C6H8O7 | 192.124 | 442.35 | 180.81 | 180.81 | 180.81 |
| Monobasic | NaH2PO4 | 119.98 | — | 111.01 | 111.01 | 111.01 |
| Sodium | | | | | | |
| Phosphate | | | | | | |
| Dextrose | C6H12O6 | 180.16 | 1343.16 | 1397.98 | 2795.96 | 1743.96 |
| Adenine | C5H5N5 | 135.13 | — | — | — | 18.19 |

| | | | | |
|---|---|---|---|---|
| Total Weight of chemicals | 2906.1 | 3029.4 | 4427.4 | 3393.6 |
| for 450 mL whole | | | | |
| blood collection (mg) | | | | |

TABLE 6B
Composition of non-limiting dry anticoagulant/preservative
formulations to be combined with 450 mL of donated whole
blood (still upper limit)

| Chemical | Chemical | MW | ACDA | CPD | CP2D | CPDA-1 |
|---|---|---|---|---|---|---|
| Name | Formula | (g/mol) | (mg) | (mg) | (mg) | (mg) |
| Sodium | C6H5Na3O7 | 258.06 | 1369 | 1527 | 1527 | 1527 |
| Citrate | | | | | | |
| Citric Acid | C6H8O7 | 192.124 | 519 | 198 | 198 | 198 |
| Monobasic | NaH2PO4 | 119.98 | — | 128 | 128 | 128 |
| Sodium | | | | | | |
| Phosphate | | | | | | |
| Dextrose | C6H12O6 | 180.16 | 1579 | 1534 | 3068 | 1919 |
| Adenine | C5H5N5 | 135.13 | — | — | — | 19 |

| | | | | |
|---|---|---|---|---|
| Total Weight of chemicals | 3467 | 3387 | 4921 | 3791 |
| for 450 mL whole blood | | | | |
| collection (mg) | | | | |

Of course, the amounts listed in the non-limiting examples set forth in Tables 3A-6B above may also vary by +/−5% and/or by +/−10% based on weight or based on the targeted blood volume.

The invention contemplates dry anticoagulant/preservative formulations other than those listed in Tables 3A-6B above, as long as the anticoagulant/preservative formulations is added to donated whole blood without the addition of any free H2O molecules.

It should also be noted that the chemicals listed in the non-limiting examples in Tables 3A-6B above are provided in their anhydrous form. For example, while sodium citrate is listed above as having the chemical formula of C₆H₅Na₃O₇ and having a molecular weight of about 258 grams per mole, a common form of sodium citrate is the dihydrate form, which as a molecular weight of about 294 grams per mole. In the event that a dihydrate form of sodium citrate is used in a dry formulation, the routinely skilled person would understand that slightly more dihydrate sodium citrate must be used in the formulation as compared to anhydrous sodium citrate because the two water molecules each add about 18 g/mole (so a total of about 36 g/mole for the two H₂O molecules).

In additional embodiments, Tables 7A-10B below provide non-limiting examples of the molar values for the indicated chemical ingredients at the lower, still lower, upper, and still upper limits for 500 mL donated whole blood and 450 mL donated whole blood.

TABLE 7A
Composition of dry anticoagulant/preservative formulations and
additives for 500 mL WB collection (milli moles lower limit)

| | | MW | ACD-A | CPD | CP2D | CPDA-1 |
|---|---|---|---|---|---|---|
| Chemical | Formula | (g/mol) | (mmol) | (mmol) | (mmol) | (mmol) |
| Sodium Citrate | C6H5Na3O7 | 258.06 | 4.03 | 4.82 | 4.82 | 4.82 |
| Citric Acid | C6H8O7 | 192.124 | 2.14 | 0.87 | 0.87 | 0.87 |
| Monobasic | NaH2PO4 | 119.98 | — | 0.86 | 0.86 | 0.86 |
| Sodium | | | | | | |
| Phosphate | | | | | | |
| Dextrose | C6H12O6 | 180.16 | 6.92 | 7.20 | 14.41 | 8.99 |
| Adenine | C5H5N5 | 135.13 | — | — | — | 0.12 |

TABLE 7B
Composition of dry anticoagulant/preservative formulations and
additives for 500 mL WB collection (milli moles still lower limit)

| | | MW | ACD-A | CPD | CP2D | CPDA-1 |
|---|---|---|---|---|---|---|
| Chemical | Formula | (g/mol) | (mmol) | (mmol) | (mmol) | (mmol) |
| Sodium | C6H5Na3O7 | 258.06 | 4.20 | 4.75 | 4.75 | 4.75 |
| Citrate | | | | | | |
| Citric Acid | C6H8O7 | 192.124 | 2.13 | 0.82 | 0.82 | 0.82 |
| Monobasic | NaH2PO4 | 119.98 | — | 0.85 | 0.85 | 0.85 |
| Sodium | | | | | | |
| Phosphate | | | | | | |
| Dextrose | C6H12O6 | 180.16 | 6.94 | 6.84 | 13.69 | 8.56 |
| Adenine | C5H5N5 | 135.13 | — | — | — | 0.10 |

TABLE 8A
Composition of dry anticoagulant/preservative formulations and
additives for 500 mL WB collection (milli moles upper limit)

| | | MW | ACD-A | CPD | CP2D | CPDA-1 |
|---|---|---|---|---|---|---|
| Chemical | Formula | (g/mol) | (mmol) | (mmol) | (mmol) | (mmol) |
| Sodium Citrate | C6H5Na3O7 | 258.06 | 4.82 | 5.77 | 5.77 | 5.77 |
| Citric Acid | C6H8O7 | 192.124 | 2.56 | 1.05 | 1.05 | 1.05 |
| Monobasic | NaH2PO4 | 119.98 | — | 1.03 | 1.03 | 1.03 |
| Sodium | | | | | | |
| Phosphate | | | | | | |
| Dextrose | C6H12O6 | 180.16 | 8.28 | 8.62 | 17.24 | 10.76 |
| Adenine | C5H5N5 | 135.13 | — | — | — | 0.15 |

TABLE 8B
Composition of dry anticoagulant/preservative formulations and
additives for 500 mL WB collection (milli moles still upper limit)

| | | MW | ACD-A | CPD | CP2D | CPDA-1 |
|---|---|---|---|---|---|---|
| Chemical | Formula | (g/mol) | (mmol) | (mmol) | (mmol) | (mmol) |
| Sodium | C6H5Na3O7 | 258.06 | 5.90 | 6.58 | 6.58 | 6.58 |
| Citrate | | | | | | |
| Citric Acid | C6H8O7 | 192.124 | 3.00 | 1.15 | 1.15 | 1.15 |
| Monobasic | NaH2PO4 | 119.98 | — | 1.19 | 1.19 | 1.19 |
| Sodium | | | | | | |
| Phosphate | | | | | | |
| Dextrose | C6H12O6 | 180.16 | 9.74 | 9.46 | 18.92 | 11.84 |
| Adenine | C5H5N5 | 135.13 | — | — | — | 0.15 |

TABLE 9 A
Composition of dry anticoagulant/preservative formulations and
additives for 450 ml WB collection (milli moles lower limit)

| | | MW | ACD-A | CPD | CP2D | CPDA-1 |
|---|---|---|---|---|---|---|
| Chemical | Formula | (g/mol) | (mmol) | (mmol) | (mmol) | (mmol) |
| Sodium | C6H5Na3O7 | 258.07165 | 3.63 | 4.34 | 4.34 | 4.34 |
| Citrate | | | | | | |
| Citric Acid | C6H8O7 | 192.12556 | 1.92 | 0.79 | 0.79 | 0.79 |
| Monobasic | NaH2PO4 | 119.97754 | — | 0.77 | 0.77 | 0.77 |
| Sodium | | | | | | |
| Phosphate | | | | | | |
| Dextrose | C6H12O6 | 180.15804 | 6.23 | 6.48 | 12.97 | 8.09 |
| Adenine | C5H5N5 | 135.12985 | — | — | — | 0.11 |

TABLE 9B
Composition of dry anticoagulant/preservative formulations and
additives for 450 ml WB collection (milli moles still lower limit)

| | | MW | ACD-A | CPD | CP2D | CPDA-1 |
|---|---|---|---|---|---|---|
| Chemical | Formula | (g/mol) | (mmol) | (mmol) | (mmol) | (mmol) |
| Sodium | C6H5Na3O7 | 258.06 | 3.86 | 4.37 | 4.75 | 4.37 |
| Citrate | | | | | | |
| Citric Acid | C6H8O7 | 192.124 | 1.96 | 0.76 | 0.76 | 0.76 |
| Monobasic | NaH2PO4 | 119.98 | — | 0.79 | 0.79 | 0.79 |
| Sodium | | | | | | |
| Phosphate | | | | | | |
| Dextrose | C6H12O6 | 180.16 | 6.38 | 6.29 | 12.57 | 7.86 |
| Adenine | C5H5N5 | 135.13 | — | — | — | 0.10 |

TABLE 10A
Composition of dry anticoagulant/preservative formulations and
additives for 450 ml WB collection (milli moles upper limit)

| | | MW | ACD-A | CPD | CP2D | CPDA-1 |
|---|---|---|---|---|---|---|
| Chemical | Formula | (g/mol) | (mmol) | (mmol) | (mmol) | (mmol) |
| Sodium | C6H5Na3O7 | 258.07165 | 4.34 | 5.19 | 5.19 | 5.19 |
| Citrate | | | | | | |
| Citric Acid | C6H8O7 | 192.12556 | 2.30 | 0.94 | 0.94 | 0.94 |
| Monobasic | NaH2PO4 | 119.97754 | — | 0.93 | 0.93 | 0.93 |
| Sodium | | | | | | |
| Phosphate | | | | | | |
| Dextrose | C6H12O6 | 180.15804 | 7.46 | 7.76 | 15.52 | 9.68 |
| Adenine | C5H5N5 | 135.12985 | — | — | — | 0.13 |

TABLE 10B
Composition of dry anticoagulant/preservative formulations and
additives for 450 ml WB collection (milli moles still upper limit)

| | | MW | ACD-A | CPD | CP2D | CPDA-1 |
|---|---|---|---|---|---|---|
| Chemical | Formula | (g/mol) | (mmol) | (mmol) | (mmol) | (mmol) |
| Sodium | C6H5Na3O7 | 258.06 | 5.30 | 5.92 | 5.92 | 5.92 |
| Citrate | | | | | | |
| Citric Acid | C6H8O7 | 192.124 | 2.70 | 1.03 | 1.03 | 1.03 |
| Monobasic | NaH2PO4 | 119.98 | — | 1.06 | 1.06 | 1.06 |
| Sodium | | | | | | |
| Phosphate | | | | | | |
| Dextrose | C6H12O6 | 180.16 | 8.76 | 8.51 | 17.02 | 10.65 |
| Adenine | C5H5N5 | 135.13 | — | — | — | 0.13 |

It shall be understood, of course, that the ingredients (and the amounts thereof) listed in Tables 3A-10B are simply exemplary, and should not be construed as limiting the dry anticoagulant/preservative formulations described herein. For example, the amount of any of the ingredients may vary by + or −(i.e., +/−) 30%, or +/−25%, or +/−20%, or +/−15%, or +/−10%, or +/−5%, or +/−1%.

It should be noted that the dry (or solid) compositions or formulations described herein yield an undiluted whole blood product with similar exogenous ingredients (e.g., added dextrose or monobasic sodium phosphate) to their conventional water-based (i.e., liquid) dilute whole blood composition. The ingredients may be further refined and adjusted to improve storage and stability of blood storage since the dry compositions described herein provide improved stability of composition due to the fact that the dry compositions (or formulations) described herein do not include free water. For example, a liquid CPD formulation is made acidic by design to prevent caramelization for the sugar components (e.g., dextrose) for long term storage. But since the sugar components in the dry formulations described herein will not caramelize, increasing the acidity of the dry formulations described herein is unnecessary. Also, some of the ingredients are added to the dry formulations and compositions described herein because these ingredients maintain or reduce the concentration gradient across the cellular components of the blood, therefore, less of the ingredient (e.g. monobasic Sodium Phosphate) is needed in the dry formulation described herein since the volume of fluid in that the cellular components are collected and/or stored in is reduced. Ingredients such as dextrose are also included in anticoagulant/preservation formulations for cellular metabolism and these ingredients are provided in the dry formulations described herein the same concentration that they are present in liquid formulations; however, the concentration of these ingredients may be adjusted based on the desired shelf-life of blood product that is intended to be transfused to a recipient patient. For example, if the dry or solid formulations or compositions as described herein are intended to be shipped to combat sites where donor whole blood may be collected and stored for fewer than, for example, seven days prior to transfusion to a patient in need (e.g., a soldier with a gunshot wound), the ingredients that play a role in cellular metabolism (e.g., dextrose) may be reduced in that dry formulation.

Thus, it will be understood that the examples described herein are not intended to limit the invention but, rather, to demonstrate dry formulations with a similar ingredient make-up and concentration as compared to the conventional liquid additives currently used. For example, the total citrate content in the dry anticoagulant/preservative formulations described herein may be reduced to provide a lower concentration of citrate but other higher values of citrate or alternative anticoagulant may also be used in the dry anticoagulant/preservative formulations described herein.

It should be noted that when the phrase “mixed with” is used in reference to a formulation or compound as described herein mixed with whole blood (or, in some instances, a blood product such as packed red blood cells), the phrase simply means that the formulation is mixed with the indicated amount of whole blood (or a blood product). The formulation may be mixed with the whole blood (or blood product) by adding the formulation to the whole blood (or blood product). For example, whole blood may be collected into a blood bag, and then the formulation, in the form of powder or granules or one or more tablets (or other forms, including one or more capsules) or one more of these forms may then be added to the blood within the blood bag. In another example, the blood bag may be manufactured such that a formulation as described herein is contained in the interior of the blood bag, and whole blood (or a blood product) is mixed with the formulation by adding the whole blood (or blood product) to the formulation-containing bag. One of the examples below described the manufacture of a blood bag containing a formulation as described herein.

The processing of whole blood into different components is well known, and one useful blood product is red blood cells collected from whole blood. For example, after mixing donated whole blood with one of the dry anticoagulant/preservative formulations described herein, the blood may be separated into a red blood cell fraction and a plasma fraction using standard methods such as centrifugation of the blood bag to separate the components by density. In some embodiments, white blood cells are removed from the whole blood prior, at the same time as, or after the separation of the blood into a red blood cell fraction and a plasma fraction. White blood cells may be removed from whole blood (or a blood product thereof) by, for example, using a leukoreduction filter or centrifugation, producing leukoreduced blood. Leukoreduction in blood banking is a well known technique in blood banking and is primarily achieved through filtration, a process where special filters remove white blood cells from blood products. Leukoreduction can occur at various stages, including immediately after collection of donor whole blood, before storage of the whole blood or blood product, or just before transfusion to the patient. Centrifugation followed by the removal of the buffy coat layer—rich in leukocytes—and, less commonly, washing with saline to eliminate residual leukocytes and plasma proteins, are additional methods employed to achieve leukoreduction. These techniques aim to reduce the risk of transfusion-related complications by minimizing the presence of white blood cells in transfused blood.

In some embodiments, platelets are removed from the whole blood prior to, at the same time as, or after the separation of the blood into a red blood cell fraction and a plasma fraction. In some embodiments, the invention provides for dry or solid compositions and formulations that may be added to platelets prepared from whole blood. In some embodiments, the invention provides for dry or solid compositions and formulations that may be added to plasma prepared from whole blood. In additional embodiments, the invention provides for dry or solid compositions and formulations that may be added to red blood cells prepared from whole blood.

Packed red blood cells are simply red blood cells from whole blood or any other source (e.g., apheresis) with about 80% of the plasma removed. One unit of donated whole blood contains between about 450 mL to about 500 mL of whole blood. The proportion of components in whole blood is approximately 55% plasma, less than or equal to 1% white blood cells and platelets, and about 44% red blood cells (i.e., totaling 100%). Since about 55% of whole blood is plasma, there is between about 248 mL to about 275 ml plasma. Removing about 80% of the plasma leaves between about 50 mL to about 55 mL plasma, which forms a layer on top of the denser layer red blood cells. The layer of red blood cells at the bottom are tightly packed, and are thus referred to as packed red blood cells. To these packed red blood cells, red blood cell additive solution is added. These RBC additive solutions, some of which are listed above in Table 2, are mixed with packed red bloods cells to create a red blood cell unit.

The quantity of RBC additive solution added to packed red blood cells is typically at a ratio of 1:4.5 of whole blood (WB) collection. Therefore, for about standard 450 mL and 500 mL WB collection about 100 ml and about 110 mL of additive is used, respectively.

However, it would be useful to not have the addition of water to red blood cell packs. Like whole blood, the manufacture of bags containing liquid red blood cell additive solution is cumbersome and costly, requiring multiple sterilizations steps. And, like blood containing liquid anticoagulant/preservative solution, blood bags containing liquid red blood cell additive solution are bulky and heavy, rendering their storage and shipping more burdensome than a blood bag containing a solid formulation that may be added to red blood cells as a red blood cell additive. In some embodiments, sterile water may be added to the blood bag containing the solid formulation prior to the addition of the red blood cells, but this addition of water may occur at the site where the blood bag is to be used, and not at the site where the blood bag is manufactured. As sterile water is used for patient care purposes (reconstitution of dry drug formulations), this would be suitable alternative.

Accordingly, in another aspect, the invention provides a solid formulation that can be added to whole blood that provides anticoagulant activity, preservative activity, as well as red blood cell additive activity. For example, APEX-A and APEX B are formulations that may be added to conventional CPD and CPDA-1 or CP2D for preservation of WB. When CPDA-1 is added to APEX-A, it creates APEX-TA. When CPDA-1 is added to APEX-B, it creates APEX-TB.

Thus, in various embodiments, the invention can be described in the examples set forth in Tables 11A and 11B, which provide examples of the lower and still lower limit of chemical components of a dry formulation (i.e., lacking free water molecules) with both anticoagulant and red blood cell additive activity for addition to 450 mL Whole Blood, where each ingredient is, for example, within ±5% of the stated value.

TABLE 11A
Composition of anticoagulants, preservatives, and other additives
for 450 mL WB collection (mg lower limit)

| | | | APEX- | APEX- | | |
|---|---|---|---|---|---|---|
| | | MW | A | B ™ | APEX-T_{A} ™ | APEX-T_{B} ™ |
| Chemical | Formula | (g/mol) | (mg) | (mg) | (mg) | (mg) |
| Sodium | C6H5Na3O7 | 258.06 | — | — | 1127 | 1127 |
| Citrate | | | | | | |
| Citric Acid | C6H8O7 | 192.124 | — | — | 146 | 146 |
| Monobasic | NaH2PO4 | 119.98 | — | — | 94 | 94 |
| Sodium | | | | | | |
| Phosphate | | | | | | |
| Dextrose | C6H12O6 | 180.16 | — | — | 1416 | 1416 |
| Adenine | C5H5N5 | 135.13 | 10 | 10 | 23 | 23 |
| D-Mannitol | C6H14O6 | 182.17 | 757 | 757 | 757 | 757 |
| Dibasic | Na2HPO4 | 141.96 | 128 | 128 | 128 | 128 |
| Sodium | | | | | | |
| Phosphate | | | | | | |
| Sodium | NaHCO3 | 84.007 | 253 | 253 | 253 | 253 |
| Bicarbonate | | | | | | |
| Guanosine | C10H13N5O5 | 283.24 | — | — | — | — |
| Sodium | C2H3NaO2 | 82.034 | 310 | 310 | 310 | 310 |
| Acetate | | | | | | |
| Magnesium | C12H10Mg3O14 | 451.13 | — | 45 | — | 45 |
| Citrate | | | | | | |
| (tribasic) | | | | | | |
| Sodium | NaCl | 58.443 | — | — | — | — |
| Chloride | | | | | | |

| | | | | |
|---|---|---|---|---|
| Total Weight of | 1458 | 1543 | 4254 | 4339 |
| chemicals for 450 | | | | |
| ml WB Collection (mg) | | | | |

TABLE 11B
Composition of anticoagulants, preservatives, and other additives
for 450 mL WB collection (mg still lower limit)

| | | | APEX- | APEX- | APEX- | APEX- |
|---|---|---|---|---|---|---|
| | | MW | A | B ™ | T_{A} ™ | T_{B} ™ |
| Chemical | Formula | (g/mol) | (mg) | (mg) | (mg) | (mg) |
| Sodium | C6H5Na3O7 | 258.06 | — | — | 1127 | 1127 |
| Citrate | | | | | | |
| Citric Acid | C6H8O7 | 192.124 | — | — | 146 | 146 |
| Monobasic | NaH2PO4 | 119.98 | — | — | 94 | 94 |
| Sodium | | | | | | |
| Phosphate | | | | | | |
| Dextrose | C6H12O6 | 180.16 | — | — | 1416 | 1416 |
| Adenine | C5H5N5 | 135.13 | 10 | 10 | 23 | 23 |
| D-Mannitol | C6H14O6 | 182.17 | 757 | 757 | 757 | 757 |
| Dibasic | Na2HPO4 | 141.96 | 128 | 128 | 128 | 128 |
| Sodium | | | | | | |
| Phosphate | | | | | | |
| Sodium | NaHCO3 | 84.007 | 253 | 253 | 253 | 253 |
| Bicarbonate | | | | | | |
| Guanosine | C10H13N5O5 | 283.24 | — | — | — | — |
| Sodium | C2H3NaO2 | 82.034 | 310 | 310 | 310 | 310 |
| Acetate | | | | | | |
| Magnesium | C6H8MgO8 | 214.41 | 64 | — | 64 | — |
| Citrate | | | | | | |
| (dibasic) | | | | | | |
| Sodium | NaCl | 58.443 | — | — | — | — |
| Chloride | | | | | | |

| | | | | |
|---|---|---|---|---|
| Total Weight of chemicals for | 1522 | 1458 | 4318 | 4254 |
| 450 ml WB Collection (mg) | | | | |

In some embodiments, Tables 12A and 12B provide non-limiting examples of the upper and still upper limits of chemical components of a dry formulation (i.e., lacking free water molecules) with anticoagulant activity, preservative activity, and red blood cell additive activity for addition to 450 mL whole blood, where each ingredient is, for example, within ±/−5% of the stated value.

TABLE 12A
Composition of anticoagulants, preservatives, and other additives
for 450 mL WB collection (mg upper limit)

| | | MW | APEX-A | APEX-B ™ | APEX-T_{A} ™ | APEX-T_{B} ™ |
|---|---|---|---|---|---|---|
| Chemical | Formula | (g/mol) | (mg) | (mg) | (mg) | (mg) |
| Sodium | C6H5Na3O7 | 258.06 | — | — | 1527 | 1527 |
| Citrate | | | | | | |
| Citric Acid | C6H8O7 | 192.124 | — | — | 198 | 198 |
| Monobasic | NaH2PO4 | 119.98 | — | — | 128 | 128 |
| Sodium | | | | | | |
| Phosphate | | | | | | |
| Dextrose | C6H12O6 | 180.16 | — | — | 1534 | 1534 |
| Adenine | C5H5N5 | 135.13 | 13 | 13 | 13 | 13 |
| D-Mannitol | C6H14O6 | 182.17 | 947 | 947 | 947 | 947 |
| Dibasic | Na2HPO4 | 141.96 | 161 | 161 | 161 | 161 |
| Sodium | | | | | | |
| Phosphate | | | | | | |
| Sodium | NaHCO3 | 84.007 | 318 | 318 | 318 | 318 |
| Bicarbonate | | | | | | |
| Guanosine | C10H13N5O5 | 283.24 | up to | up to 85 | up to 85 | up to 85 |
| | | | 85 | | | |
| Sodium | C2H3NaO2 | 82.034 | 388 | 388 | 388 | 388 |
| Acetate | | | | | | |
| Magnesium | C12H10Mg3O14 | 451.13 | — | 107 | — | 107 |
| Citrate | | | | | | |
| (tribasic) | | | | | | |
| Sodium | NaCl | 58.443 | — | — | — | — |
| Chloride | | | | | | |

| | | | | |
|---|---|---|---|---|
| Total Weight of chemicals for 450 ml | 1827 | 1884 | 5214 | 5271 |
| WB Collection (mg) | up to | up to | up to 5299 | up to 5356 |
| | 1912 | 1969 | | |

TABLE 12B
Composition of anticoagulants, preservatives, and other additives
for 450 mL WB collection (mg still upper limit)

| | | MW | APEX-A | APEX-B ™ | APEX-T_{A} ™ | APEX-T_{B} ™ |
|---|---|---|---|---|---|---|
| Chemical | Formula | (g/mol) | (mg) | (mg) | (mg) | (mg) |
| Sodium | C6H5Na3O7 | 258.06 | — | — | 1527 | 1527 |
| Citrate | | | | | | |
| Citric Acid | C6H8O7 | 192.124 | — | — | 198 | 198 |
| Monobasic | NaH2PO4 | 119.98 | — | — | 128 | 128 |
| Sodium | | | | | | |
| Phosphate | | | | | | |
| Dextrose | C6H12O6 | 180.16 | — | — | 1534 | 1534 |
| Adenine | C5H5N5 | 135.13 | 13 | 13 | 13 | 13 |
| D-Mannitol | C6H14O6 | 182.17 | 947 | 947 | 947 | 947 |
| Dibasic | Na2HPO4 | 141.96 | 161 | 161 | 161 | 161 |
| Sodium | | | | | | |
| Phosphate | | | | | | |
| Sodium | NaHCO3 | 84.007 | 318 | 318 | 318 | 318 |
| Bicarbonate | | | | | | |
| Guanosine | C10H13N5O5 | 283.24 | up to 85 | up to 85 | up to 85 | up to 85 |
| Sodium | C2H3NaO2 | 82.034 | 388 | 388 | 388 | 388 |
| Acetate | | | | | | |
| Magnesium | C6H8MgO8 | 214.41 | 57 | — | 57 | — |
| Citrate | | | | | | |
| (dibasic) | | | | | | |
| Sodium | NaCl | 58.443 | — | — | — | — |
| Chloride | | | | | | |
| Total Weight of chemicals | | | 1909 | 1827 | 5296 | 5214 |
| for 450 ml WB Collection (mg) | | | up to | up to | up to 5381 | up to 5299 |
| | | | 1994 | 1912 | | |

In further embodiments, Tables 13A and 13B provide non-limiting examples of the lower and still lower limit of chemical components of a dry formulation (i.e., lacking free water molecules) with anticoagulant, preservative, and red blood cell additive activity for addition to 500 mL whole blood, where each ingredient is, for example, within ±5% of the stated value.

TABLE 13A
Composition of anticoagulants, preservatives, and other additives for 500 ml WB collection (mg lower limit)

| | | | APEX- | | | |
|---|---|---|---|---|---|---|
| | | MW | A | APEX-B™ | APEX-T_{A}™ | APEX-T_{B}™ |
| Chemical | Formula | (g/mol) | (mg) | (mg) | (mg) | (mg) |
| Sodium | C6H5Na3O7 | 258.06 | — | — | 1225 | 1225 |
| Citrate | | | | | | |
| Citric Acid | C6H8O7 | 192.124 | — | — | 157 | 157 |
| Monobasic | NaH2PO4 | 119.98 | — | — | 101 | 101 |
| Sodium | | | | | | |
| Phosphate | | | | | | |
| Dextrose | C6H12O6 | 180.16 | — | — | 1542 | 1542 |
| Adenine | C5H5N5 | 135.13 | 10 | 10 | 25 | 25 |
| D-Mannitol | C6H14O6 | 182.17 | 832 | 832 | 832 | 832 |
| Dibasic | Na2HPO4 | 141.96 | 140 | 140 | 140 | 140 |
| Sodium | | | | | | |
| Phosphate | | | | | | |
| Sodium | NaHCO3 | 84.007 | 278 | 278 | 278 | 278 |
| Bicarbonate | | | | | | |
| Guanosine | C10H13N5O5 | 283.24 | — | — | — | — |
| Sodium | C2H3NaO2 | 82.034 | 340 | 340 | 340 | 340 |
| Acetate | | | | | | |
| Magnesium | C12H10Mg3O14 | 451.13 | — | 49 | — | 49 |
| Citrate | | | | | | |
| (tribasic) | | | | | | |
| Sodium | NaCl | 58.443 | — | — | — | — |
| Chloride | | | | | | |

| | | | | |
|---|---|---|---|---|
| Total weight of chemicals for 500 ml WB | 1600 | 1649 | 4640 | 4689 |
| Collection (mg) | | | | |

TABLE 13B
Composition of anticoagulants, preservatives, and other additives for 500 ml WB collection (mg still lower limit)

| | | MW | APEX-A | APEX-B™ | APEX-T_{A}™ | APEX-T_{B}™ |
|---|---|---|---|---|---|---|
| Chemical | Formula | (g/mol) | (mg) | (mg) | (mg) | (mg) |
| Sodium | C6H5Na3O7 | 258.06 | — | — | 1225 | 1225 |
| Citrate | | | | | | |
| Citric Acid | C6H8O7 | 192.124 | — | — | 157 | 157 |
| Monobasic | NaH2PO4 | 119.98 | — | — | 101 | 101 |
| Sodium | | | | | | |
| Phosphate | | | | | | |
| Dextrose | C6H12O6 | 180.16 | — | — | 1542 | 1542 |
| Adenine | C5H5N5 | 135.13 | 10 | 10 | 25 | 25 |
| D-Mannitol | C6H14O6 | 182.17 | 832 | 832 | 832 | 832 |
| Dibasic | Na2HPO4 | 141.96 | 140 | 140 | 140 | 140 |
| Sodium | | | | | | |
| Phosphate | | | | | | |
| Sodium | NaHCO3 | 84.007 | 278 | 278 | 278 | 278 |
| Bicarbonate | | | | | | |
| Guanosine | C10H13N5O5 | 283.24 | — | — | — | — |
| Sodium | C2H3NaO2 | 82.034 | 340 | 340 | 340 | 340 |
| Acetate | | | | | | |
| Magnesium | C6H8MgO8 | 214.41 | 70 | — | 70 | — |
| Citrate | | | | | | |
| (dibasic) | | | | | | |
| Sodium | NaCl | 58.443 | — | — | — | — |
| Chloride | | | | | | |

| | | | | |
|---|---|---|---|---|
| Total weight of chemicals for 500 ml WB | 1670 | 1600 | 4710 | 4640 |
| Collection (mg) | | | | |

In still further embodiments, Tables 14A and 14B provide non-limiting examples of the upper and still upper limits of chemical components of a dry formulation (i.e., lacking free water molecules) with anticoagulant, preservative, and red blood cell additive activity for addition to 500 mL Whole Blood, where each ingredient is, for example, within ±5% of the stated value.

TABLE 14A
Composition of anticoagulants, preservatives, and other additives for 500 mL WB collection (mg upper limit)

| | | | | | | APEX- |
|---|---|---|---|---|---|---|
| | | MW | APEX-A | APEX-B™ | APEX-T_{A}™ | T_{B}™ |
| Chemical | Formula | (g/mol) | (mg) | (mg) | (mg) | (mg) |
| Sodium | C6H5Na3O7 | 258.06 | — | — | 1699 | 1699 |
| Citrate | | | | | | |
| Citric Acid | C6H8O7 | 192.124 | — | — | 221 | 221 |
| Monobasic | NaH2PO4 | 119.98 | — | — | 143 | 143 |
| Sodium | | | | | | |
| Phosphate | | | | | | |
| Dextrose | C6H12O6 | 180.16 | — | — | 1705 | 1705 |
| Adenine | C5H5N5 | 135.13 | 15 | 15 | 15 | 15 |
| D-Mannitol | C6H14O6 | 182.17 | 1053 | 1053 | 1053 | 1053 |
| Dibasic | Na2HPO4 | 141.96 | 179 | 179 | 179 | 179 |
| Sodium | | | | | | |
| Phosphate | | | | | | |
| Sodium | NaHCO3 | 84.007 | 353 | 353 | 353 | 353 |
| Bicarbonate | | | | | | |
| Guanosine | C10H13N5O5 | 283.24 | up to 94 | up to 94 | up to 94 | up to 94 |
| Sodium | C2H3NaO2 | 82.034 | 431 | 431 | 431 | 431 |
| Acetate | | | | | | |
| Magnesium | C12H10Mg3O14 | 451.13 | — | 64 | — | 64 |
| Citrate | | | | | | |
| (tribasic) | | | | | | |
| Sodium | NaCl | 58.443 | — | — | — | — |
| Chloride | | | | | | |
| Total weight of chemicals for 500 ml WB | | | 2031 | 2095 | 5799 | 5863 |
| Collection | | | up to | up to | up to | up to |
| | | | 2125 | 2189 | 5893 | 5957 |

TABLE 14B
Composition of anticoagulants, preservatives, and other additives for 500 mL WB collection (mg still upper limit)

| | | | | | | APEX- |
|---|---|---|---|---|---|---|
| | | MW | APEX-A | APEX-B™ | APEX-T_{A}™ | T_{B}™ |
| Chemical | Formula | (g/mol) | (mg) | (mg) | (mg) | (mg) |
| Sodium | C6H5Na3O7 | 258.06 | — | — | 1699 | 1699 |
| Citrate | | | | | | |
| Citric Acid | C6H8O7 | 192.124 | — | — | 221 | 221 |
| Monobasic | NaH2PO4 | 119.98 | — | — | 143 | 143 |
| Sodium | | | | | | |
| Phosphate | | | | | | |
| Dextrose | C6H12O6 | 180.16 | — | — | 1705 | 1705 |
| Adenine | C5H5N5 | 135.13 | 15 | 15 | 15 | 15 |
| D-Mannitol | C6H14O6 | 182.17 | 1053 | 1053 | 1053 | 1053 |
| Dibasic | Na2HPO4 | 141.96 | 179 | 179 | 179 | 179 |
| Sodium | | | | | | |
| Phosphate | | | | | | |
| Sodium | NaHCO3 | 84.007 | 353 | 353 | 353 | 353 |
| Bicarbonate | | | | | | |
| Guanosine | C10H13N5O5 | 283.24 | up to 94 | up to 94 | up to 94 | up to 94 |
| Sodium | C2H3NaO2 | 82.034 | 431 | 431 | 431 | 431 |
| Acetate | | | | | | |
| Magnesium | C6H8MgO8 | 214.41 | 91 | — | 91 | — |
| Citrate | | | | | | |
| (dibasic) | | | | | | |
| Sodium | NaCl | 58.443 | | | — | — |
| Chloride | | | | | | |
| Total weight of chemicals for 500 ml WB | | | 2122 | 2031 | 5890 | 5799 |
| Collection | | | up to | up to | up to | up to |
| | | | 2216 | 2125 | 5984 | 5893 |

In yet further embodiments, Tables 15A and 15B provide non-limiting examples of the lower and still lower limits of the moles of chemical components of a dry formulation (i.e., lacking free water molecules) with anticoagulant, preservative, and red blood cell additive activity for addition to 450 ml whole blood (“WB”), where each ingredient is, for example, within ±5% of the stated value.

TABLE 15A
Composition of anticoagulants, preservatives, and other additives for 450 mL WB collection (milli moles lower limit)

| | | | | APEX- | APEX- | APEX- |
|---|---|---|---|---|---|---|
| | | MW | APEX-A | B™ | T_{A}™ | T_{B}™ |
| Chemical | Formula | (g/mol) | (mmol) | (mmol) | (mmol) | (mmol) |
| Sodium Citrate | C6H5Na3O7 | 258.06 | — | — | 4.37 | 4.37 |
| Citric Acid | C6H8O7 | 192.124 | — | — | 0.76 | 0.76 |
| Monobasic | NaH2PO4 | 119.98 | — | — | 0.79 | 0.79 |
| Sodium | | | | | | |
| Phosphate | | | | | | |
| Dextrose | C6H12O6 | 180.16 | — | — | 7.86 | 7.86 |
| Adenine | C5H5N5 | 135.13 | 0.08 | 0.08 | 0.18 | 0.18 |
| D-Mannitol | C6H14O6 | 182.17 | 4.16 | 4.16 | 4.16 | 4.16 |
| Dibasic Sodium | Na2HPO4 | 141.96 | 0.91 | 0.91 | 0.91 | 0.91 |
| Phosphate | | | | | | |
| Sodium | NaHCO3 | 84.007 | 3.02 | 3.02 | 3.02 | 3.02 |
| Bicarbonate | | | | | | |
| Guanosine | C10H13N5O5 | 283.24 | — | — | — | — |
| Sodium Acetate | C2H3NaO2 | 82.034 | 3.78 | 3.78 | 3.78 | 3.78 |
| Magnesium | C12H10Mg3O14 | 451.13 | — | 0.10 | — | 0.10 |
| Citrate (tribasic) | | | | | | |
| Sodium | NaCl | 58.443 | — | — | — | — |
| Chloride | | | | | | |

| | | | | |
|---|---|---|---|---|
| Total moles of chemicals for 450 ml WB | 11.95 | 12.05 | 25.83 | 25.93 |
| Collection | | | | |

TABLE 15B
Composition of anticoagulants, preservatives, and other additives for 450 mL WB collection (milli moles still lower limit)

| | | | | APEX- | APEX- | |
|---|---|---|---|---|---|---|
| | | MW | APEX-A | B™ | T_{A}™ | APEX-T_{B}™ |
| Chemical | Formula | (g/mol) | (mmol) | (mmol) | (mmol) | (mmol) |
| Sodium | C6H5Na3O7 | 258.06 | — | — | 4.37 | 4.37 |
| Citrate | | | | | | |
| Citric Acid | C6H8O7 | 192.124 | — | — | 0.76 | 0.76 |
| Monobasic | NaH2PO4 | 119.98 | — | — | 0.79 | 0.79 |
| Sodium | | | | | | |
| Phosphate | | | | | | |
| Dextrose | C6H12O6 | 180.16 | — | — | 7.86 | 7.86 |
| Adenine | C5H5N5 | 135.13 | 0.08 | 0.08 | 0.18 | 0.18 |
| D-Mannitol | C6H14O6 | 182.17 | 4.16 | 4.16 | 4.16 | 4.16 |
| Dibasic | Na2HPO4 | 141.96 | 0.91 | 0.91 | 0.91 | 0.91 |
| Sodium | | | | | | |
| Phosphate | | | | | | |
| Sodium | NaHCO3 | 84.007 | 3.02 | 3.02 | 3.02 | 3.02 |
| Bicarbonate | | | | | | |
| Guanosine | C10H13N5O5 | 283.24 | — | — | — | — |
| Sodium | C2H3NaO2 | 82.034 | 3.78 | 3.78 | 3.78 | 3.78 |
| Acetate | | | | | | |
| Magnesium | C6H8MgO8 | 214.41 | 0.30 | — | 0.30 | — |
| Citrate | | | | | | |
| (dibasic) | | | | | | |
| Sodium | NaCl | 58.443 | — | — | — | — |
| Chloride | | | | | | |

| | | | | |
|---|---|---|---|---|
| Total moles of chemicals for 450 ml WB | 12.25 | 11.95 | 26.13 | 25.83 |
| Collection | | | | |

In further embodiments, Tables 16A and 16B provide non-limiting examples of the upper and still upper limits of the moles of chemical components of a dry formulation (i.e., lacking free water molecules) with anticoagulant, preservative, and red blood cell additive activity for addition to 450 mL Whole Blood, where each ingredient is, for example, within ±5% of the stated value.

TABLE 16A
Composition of anticoagulants, preservatives, and other additives
for 450 mL WB collection (milli moles upper limit)

| | | | | | APEX- | APEX- |
|---|---|---|---|---|---|---|
| | | MW | APEX-A | APEX-B™ | TA™ | TR™ |
| Chemical | Formula | (g/mol) | (mmol) | (mmol) | (mmol) | (mmol) |
| Sodium | C6H5Na3O7 | 258.06 | — | — | 5.92 | 5.92 |
| Citrate | | | | | | |
| Citric Acid | C6H8O7 | 192.124 | — | — | 1.03 | 1.03 |
| Monobasic | NaH2PO4 | 119.98 | — | — | 1.06 | 1.06 |
| Sodium | | | | | | |
| Phosphate | | | | | | |
| Dextrose | C6H12O6 | 180.16 | — | — | 8.51 | 8.51 |
| Adenine | C5H5N5 | 135.13 | 0.09 | 0.09 | 0.09 | 0.09 |
| D-Mannitol | C6H14O6 | 182.17 | 5.20 | 5.20 | 5.20 | 5.20 |
| Dibasic | Na2HPO4 | 141.96 | 1.13 | 1.13 | 1.13 | 1.13 |
| Sodium | | | | | | |
| Phosphate | | | | | | |
| Sodium | NaHCO3 | 84.007 | 3.78 | 3.78 | 3.78 | 3.78 |
| Bicarbonate | | | | | | |
| Guanosine | C10H13N5O5 | 283.24 | up to 0.3 | up to 0.3 | up to 0.3 | up to 0.3 |
| Sodium | C2H3NaO2 | 82.034 | 4.73 | 4.73 | 4.73 | 4.73 |
| Acetate | | | | | | |
| Magnesium | C12H10Mg3O14 | 451.13 | — | 0.13 | — | 0.13 |
| Citrate | | | | | | |
| (tribasic) | | | | | | |
| Sodium | NaCl | 58.443 | — | — | — | — |
| Chloride | | | | | | |

| | | | | |
|---|---|---|---|---|
| Total moles of chemicals for 450 ml WB | 14.93 up | 15.06 up | 31.45 up | 31.58 up |
| Collection | to 15.23 | to 15.36 | to 31.75 | to 31.88 |

TABLE 16B
Composition of anticoagulants, preservatives, and other additives
for 450 mL WB collection (milli moles still upper limit)

| | | | | | APEX- | APEX- |
|---|---|---|---|---|---|---|
| | | | | | T_{A}™ | T_{B}™ |
| Chemical | Formula | MW | APEX-A | APEX-B™ | (mmol) | (mmol) |
| Sodium | C6H5Na3O7 | 258.06 | — | — | 5.92 | 5.92 |
| Citrate | | | | | | |
| Citric Acid | C6H8O7 | 192.124 | — | — | 1.03 | 1.03 |
| Monobasic | NaH2PO4 | 119.98 | — | — | 1.06 | 1.06 |
| Sodium | | | | | | |
| Phosphate | | | | | | |
| Dextrose | C6H12O6 | 180.16 | — | — | 8.51 | 8.51 |
| Adenine | C5H5N5 | 135.13 | 0.09 | 0.09 | 0.09 | 0.09 |
| D-Mannitol | C6H14O6 | 182.17 | 5.20 | 5.20 | 5.20 | 5.20 |
| Dibasic | Na2HPO4 | 141.96 | 1.13 | 1.13 | 1.13 | 1.13 |
| Sodium | | | | | | |
| Phosphate | | | | | | |
| Sodium | NaHCO3 | 84.007 | 3.78 | 3.78 | 3.78 | 3.78 |
| Bicarbonate | | | | | | |
| Guanosine | C10H13N5O5 | 283.24 | up to 0.3 | up to 0.3 | up to 0.3 | up to 0.3 |
| Sodium | C2H3NaO2 | 82.034 | 4.73 | 4.73 | 4.73 | 4.73 |
| Acetate | | | | | | |
| Magnesium | C6H8MgO8 | 214.41 | 0.38 | — | 0.38 | — |
| Citrate | | | | | | |
| (dibasic) | | | | | | |
| Sodium | NaCl | 58.443 | — | — | — | — |
| Chloride | | | | | | |

| | | | | |
|---|---|---|---|---|
| Total moles of chemicals for 450 ml WB | 15.31 | 14.93 | 31.83 | 31.45 |
| Collection | up to | Up to | up to | up to |
| | 15.61 | 15.23 | 32.13 | 31.75 |

In further embodiments, Tables 17A and 17B provide non-limiting examples of the lower and still lower limits of the moles of chemical components of a dry formulation (i.e., lacking free water molecules) with anticoagulant activity, preservative activity, and red blood cell additive activity for addition to 500 mL Whole Blood, where each ingredient is, for example, within ±5% of the stated value.

TABLE 17A
Composition of anticoagulant, preservatives, and other additives
for 500 mL WB collection (milli moles lower limit)

| | | | | | APEX- | APEX- |
|---|---|---|---|---|---|---|
| | | MW | APEX-A | APEX-B™ | T_{A}™ | T_{B}™ |
| Chemical | Formula | (g/mol) | (mmol) | (mmol) | (mmol) | (mmol) |
| Sodium Citrate | C6H5Na3O7 | 258.06 | — | — | 4.75 | 4.75 |
| Citric Acid | C6H8O7 | 192.124 | — | — | 0.82 | 0.82 |
| Monobasic | NaH2PO4 | 119.98 | — | — | 0.85 | 0.85 |
| Sodium | | | | | | |
| Phosphate | | | | | | |
| Dextrose | C6H12O6 | 180.16 | — | — | 8.56 | 8.56 |
| Adenine | C5H5N5 | 135.13 | 0.08 | 0.08 | 0.19 | 0.19 |
| D-Mannitol | C6H14O6 | 182.17 | 4.57 | 4.57 | 4.57 | 4.57 |
| Dibasic | Na2HPO4 | 141.96 | 0.99 | 0.99 | 0.99 | 0.99 |
| Sodium | | | | | | |
| Phosphate | | | | | | |
| Sodium | NaHCO3 | 84.007 | 3.32 | 3.32 | 3.32 | 3.32 |
| Bicarbonate | | | | | | |
| Guanosine | C10H13N5O5 | 283.24 | — | — | — | — |
| Sodium | C2H3NaO2 | 82.034 | 4.15 | 4.15 | 4.15 | 4.15 |
| Acetate | | | | | | |
| Magnesium | C12H10Mg3O14 | 451.13 | — | 0.11 | — | 0.11 |
| Citrate | | | | | | |
| (tribasic) | | | | | | |
| Sodium | NaCl | 58.443 | — | — | — | — |
| Chloride | | | | | | |

| | | | | |
|---|---|---|---|---|
| Total moles of chemicals for 500 ml WB | 13.11 | 13.22 | 28.2 | 28.31 |
| Collection | | | | |

TABLE 17B
Composition of anticoagulants, preservatives, and other additives
for 500 mL WB collection (milli moles still lower limit)

| | | | | | APEX- | APEX- |
|---|---|---|---|---|---|---|
| | | MW | APEX-A | APEX-B™ | T_{A}™ | T_{B}™ |
| Chemical | Formula | (g/mol) | (mmol) | (mmol) | (mmol) | (mmol) |
| Sodium Citrate | C6H5Na3O7 | 258.06 | — | — | 4.75 | 4.75 |
| Citric Acid | C6H8O7 | 192.124 | — | — | 0.82 | 0.82 |
| Monobasic | NaH2PO4 | 119.98 | — | — | 0.85 | 0.85 |
| Sodium | | | | | | |
| Phosphate | | | | | | |
| Dextrose | C6H12O6 | 180.16 | — | — | 8.56 | 8.56 |
| Adenine | C5H5N5 | 135.13 | 0.08 | 0.08 | 0.19 | 0.19 |
| D-Mannitol | C6H14O6 | 182.17 | 4.57 | 4.57 | 4.57 | 4.57 |
| Dibasic | Na2HPO4 | 141.96 | 0.99 | 0.99 | 0.99 | 0.99 |
| Sodium | | | | | | |
| Phosphate | | | | | | |
| Sodium | NaHCO3 | 84.007 | 3.32 | 3.32 | 3.32 | 3.32 |
| Bicarbonate | | | | | | |
| Guanosine | C10H13N5O5 | 283.24 | — | — | — | — |
| Sodium | C2H3NaO2 | 82.034 | 4.15 | 4.15 | 4.15 | 4.15 |
| Acetate | | | | | | |
| Magnesium | C6H8MgO8 | 214.41 | 0.33 | — | 0.33 | — |
| Citrate | | | | | | |
| (dibasic) | | | | | | |
| Sodium | NaCl | 58.443 | — | — | — | — |
| Chloride | | | | | | |

| | | | | |
|---|---|---|---|---|
| Total moles of chemicals for 500 ml WB | 13.44 | 13.11 | 28.53 | 28.20 |
| Collection | | | | |

In further embodiments, Tables 18A and 18B provide non-limiting examples of the upper and still upper limit of the moles of chemical components of a dry formulation (i.e., lacking free water molecules) with anticoagulant, preservative and red blood cell additive activity for addition to 500 mL Whole Blood, where each ingredient is, for example, within ±5% of the stated value.

TABLE 18A
Composition of anticoagulants, preservatives, and other additives
for 500 mL WB collection (milli moles upper limit)

| | | | | APEX- | APEX- | APEX- |
|---|---|---|---|---|---|---|
| | | MW | APEX-A | B™ | TA™ | TB™ |
| Chemical | Formula | (g/mol) | (mmol) | (mmol) | (mmol) | (mmol) |
| Sodium Citrate | C6H5Na3O7 | 258.06 | — | — | 6.58 | 6.58 |
| Citric Acid | C6H8O7 | 192.124 | — | — | 1.15 | 1.15 |
| Monobasic | NaH2PO4 | 119.98 | — | — | 1.19 | 1.19 |
| Sodium | | | | | | |
| Phosphate | | | | | | |
| Dextrose | C6H12O6 | 180.16 | — | — | 9.46 | 9.46 |
| Adenine | C5H5N5 | 135.13 | 0.11 | 0.11 | 0.11 | 0.11 |
| D-Mannitol | C6H14O6 | 182.17 | 5.78 | 5.78 | 5.78 | 5.78 |
| Dibasic Sodium | Na2HPO4 | 141.96 | 1.26 | 1.26 | 1.26 | 1.26 |
| Phosphate | | | | | | |
| Sodium | NaHCO3 | 84.007 | 4.20 | 4.20 | 4.20 | 4.20 |
| Bicarbonate | | | | | | |
| Guanosine | C10H13N5O5 | 283.24 | up to | up to | up to | up to |
| | | | 0.33 | 0.33 | 0.33 | 0.33 |
| Sodium Acetate | C2H3NaO2 | 82.034 | 5.25 | 5.25 | 5.25 | 5.25 |
| Magnesium | C12H10Mg3O14 | 451.13 | — | 0.14 | — | 0.14 |
| Citrate (tribasic) | | | | | | |
| Sodium | NaCl | 58.443 | — | — | — | — |
| Chloride | | | | | | |

| | | | | |
|---|---|---|---|---|
| Total moles of chemicals for 500 ml WB | 16.60 | 16.74 | 34.98 | 35.12 |
| Collection | up to | up to | up to | up to |
| | 16.93 | 17.07 | 35.31 | 35.45 |

TABLE 18B
Composition of anticoagulants, preservatives, and other additives
for 500 mL WB collection (milli moles upper limit)

| | | | | | APEX- | APEX- |
|---|---|---|---|---|---|---|
| | | MW | APEX-A | APEX-B™ | T_{A}™ | T_{B}™ |
| Chemical | Formula | (g/mol) | (mmol) | (mmol) | (mmol) | (mmol) |
| Sodium | C6H5Na3O7 | 258.06 | — | — | 6.58 | 6.58 |
| Citrate | | | | | | |
| Citric Acid | C6H8O7 | 192.124 | — | — | 1.15 | 1.15 |
| Monobasic | NaH2PO4 | 119.98 | — | — | 1.19 | 1.19 |
| Sodium | | | | | | |
| Phosphate | | | | | | |
| Dextrose | C6H12O6 | 180.16 | — | — | 9.46 | 9.46 |
| Adenine | C5H5N5 | 135.13 | 0.11 | 0.11 | 0.11 | 0.11 |
| D-Mannitol | C6H14O6 | 182.17 | 5.78 | 5.78 | 5.78 | 5.78 |
| Dibasic | Na2HPO4 | 141.96 | 1.26 | 1.26 | 1.26 | 1.26 |
| Sodium | | | | | | |
| Phosphate | | | | | | |
| Sodium | NaHCO3 | 84.007 | 4.20 | 4.20 | 4.20 | 4.20 |
| Bicarbonate | | | | | | |
| Guanosine | C10H13N5O5 | 283.24 | up to | up to | up to | up to |
| | | | 0.33 | 0.33 | 0.33 | 0.33 |
| Sodium | C2H3NaO2 | 82.034 | 5.25 | 5.25 | 5.25 | 5.25 |
| Acetate | | | | | | |
| Magnesium | C6H8MgO8 | 214.41 | 0.42 | — | 0.42 | — |
| Citrate | | | | | | |
| (dibasic) | | | | | | |
| Sodium | NaCl | 58.443 | — | — | — | — |
| Chloride | | | | | | |

| | | | | |
|---|---|---|---|---|
| Total moles of chemicals for 500 ml WB | 17.02 | 16.60 | 35.40 | 34.98 |
| Collection | up to | up to | up to | up to |
| | 17.35 | 16.93 | 35.73 | 35.31 |

It shall be understood, of course, that the ingredients (and the amounts thereof) listed in Tables 11A-18B are simply exemplary, and should not be construed as limiting the dry anticoagulant/preservative formulations described herein. For example, the amount of any of the ingredients may vary by + or −(i.e., +/−) 30%, or +/−25%, or +/−20%, or +/−15%, or +/−10%, or +/−5%, or +/−1%.

In the non-limiting embodiments of the dry formulations described herein where guanosine or other nucleotides or nucleosides are included, it should be noted that the addition of guanosine and/or other nucleotides/nucleosides is not limited to red blood cell additive formulations and may be added to dry whole blood anticoagulant/preservative formulations as specified in the Tables herein. The addition of other ingredients such as amino acids and amino acid derivatives to improve storage may be considered in the formulations described herein. For example, a non-limiting formulation of the invention may include L-Carnitine which may act as a red blood cell membrane protectant. In some embodiments, the quantity of L-carnitine to be added may be up to 0.7 millimoles for 450 ml whole blood collection and 0.77 millimoles for 500 mL whole blood collection in, for example, the Tables above.

In yet further aspects, the invention provides dry formulations of red blood cell additives. If 500 mL whole blood is collected from an adult with an average hematocrit (which is 44%), the red blood cell volume is about 220 mL and the volume of plasma in that 500 mL is about 280 mL. With a liquid formulation of red cell blood additives, typically 110 mL of a liquid red blood cell additive are added to the red blood cells from 500 mL donated whole blood. Likewise, if only 450 mL is whole blood is collected, 200 ml are red blood cells, to which 100 ml liquid red blood cell additive is added. Of course, by adding liquid red blood cell additive to the packed red blood cells, the red blood cell population is diluted.

Accordingly, in some embodiments, the invention further provides dry formulations of red blood cell additives. Tables 19A-19C provide non-limiting examples of the mass of the indicated ingredients present in various dry red blood cell additive formulations in terms of microgram of ingredient per mL whole blood collected (Table 19A), grams of ingredient per 450 mL of whole blood collected (Table 19B), and grams of ingredient per 500 mL whole blood collected (Table 19 C).

TABLE 19A

| | | microgram per ml of Whole Blood Collected | | | | | | |
|---|---|---|---|---|---|---|---|---|
| Chemical | MW | CPD | CPDA-1 | AS-1 | AS-3 | AS-5 | AS-7 | APEX |
| Adenine | 135.13 | | 38.5 | 60.06 | 66 | 66 | 60.06 | 65.52 |
| (USP), | | | | | | | | |
| C5H5N5 | | | | | | | | |
| Citric Acid | 192.12 | 418.6 | 418.6 | | 84.48 | | | 418.8 |
| Monohydrate | | | | | | | | |
| (USP), | | | | | | | | |
| C6H8O7 | | | | | | | | |
| Sodium | 119.98 | 270.2 | 270.2 | | 528 | | | 268.8 |
| Dihydrogen | | | | | | | | |
| Phosphate | | | | | | | | |
| Monohydrate | | | | | | | | |
| (USP), | | | | | | | | |
| NaH2PO4 | | | | | | | | |
| Tri-Sodium | 258.07 | 3234 | 3234 | | 1135.2 | | | 3228 |
| Citrate | | | | | | | | |
| Dihydrate | | | | | | | | |
| (USP), | | | | | | | | |
| C6H5Na3O7 | | | | | | | | |
| D(+)-Glucose | 180.16 | 3248 | 4060 | 4400 | 2200 | 1801.8 | 3163.6 | 4053.6 |
| Monohydrate | | | | | | | | |
| (USP), | | | | | | | | |
| C6H12O6 | | | | | | | | |
| D(-)- | 182.17 | | | 1650 | | 1155 | 2200 | 1999.2 |
| Mannitol | | | | | | | | |
| (USP), | | | | | | | | |
| C6H14O6 | | | | | | | | |
| Di-Sodium | 141.96 | | | | | | 374 | 340.8 |
| Hydrogen | | | | | | | | |
| Phosphate | | | | | | | | |
| Anhydrous | | | | | | | | |
| (USP), | | | | | | | | |
| Na2HPO4 | | | | | | | | |
| Sodium | 84.01 | | | | | | 479.6 | 672 |
| Hydrogen | | | | | | | | |
| Carbonate | | | | | | | | |
| (USP), | | | | | | | | |
| NaHCO3 | | | | | | | | |
| Sodium | 82.03 | | | | | | | 820.8 |
| Acetate, | | | | | | | | |
| Anhydrous | | | | | | | | |
| (USP), | | | | | | | | |
| C2H3NaO2 | | | | | | | | |
| Magnesium | 214.41 | | | | | | | 172.8 |
| Citrate, | | | | | | | | |
| C6H6MgO7 | | | | | | | | |
| Magnesium | 451.12 | | | | | | | |
| Citrate, | | | | | | | | |
| Anhydrous | | | | | | | | |
| (USP), | | | | | | | | |
| C12H10Mg3 | | | | | | | | |
| O14 | | | | | | | | |
| NaCl | 58.44 | | | 1980 | 902 | 1929.4 | | |

TABLE 19B

| | | Mass for 450 mL WB Collection (g) | | | | | | |
|---|---|---|---|---|---|---|---|---|
| Chemical | MW | CPD | CPDA-1 | AS-1 | AS-3 | AS-5 | AS-7 | APEX |
| Adenine | 135.13 | | 0.0173 | 0.027 | 0.0297 | 0.0297 | 0.027 | 0.0295 |
| (USP), | | | | | | | | |
| C5H5N5 | | | | | | | | |
| Citric Acid | 192.12 | 0.188 | 0.188 | | 0.038 | | | 0.188 |
| Monohydrate | | | | | | | | |
| (USP), | | | | | | | | |
| C6H8O7 | | | | | | | | |
| Sodium | 119.98 | 0.122 | 0.122 | | 0.238 | | | 0.121 |
| Dihydrogen | | | | | | | | |
| Phosphate | | | | | | | | |
| Monohydrate | | | | | | | | |
| (USP), | | | | | | | | |
| NaH2PO4 | | | | | | | | |
| Tri-Sodium | 258.07 | 1.46 | 1.46 | | 0.511 | | | 1.45 |
| Citrate | | | | | | | | |
| Dihydrate | | | | | | | | |
| (USP), | | | | | | | | |
| C6H5Na3O7 | | | | | | | | |
| D(+)-Glucose | 180.16 | 1.46 | 1.83 | 1.98 | 0.99 | 0.811 | 1.42 | 1.82 |
| Monohydrate | | | | | | | | |
| (USP), | | | | | | | | |
| C6H12O6 | | | | | | | | |
| D(-)- | 182.17 | | | 0.743 | | 0.52 | 0.99 | 0.9 |
| Mannitol | | | | | | | | |
| (USP), | | | | | | | | |
| C6H14O6 | | | | | | | | |
| Di-Sodium | 141.96 | | | | | | 0.168 | 0.153 |
| Hydrogen | | | | | | | | |
| Phosphate | | | | | | | | |
| Anhydrous | | | | | | | | |
| (USP), | | | | | | | | |
| Na2HPO4 | | | | | | | | |
| Sodium | 84.01 | | | | | | 0.216 | 0.302 |
| Hydrogen | | | | | | | | |
| Carbonate | | | | | | | | |
| (USP), | | | | | | | | |
| NaHCO3 | | | | | | | | |
| Sodium | 82.03 | | | | | | | 0.369 |
| Acetate, | | | | | | | | |
| Anhydrous | | | | | | | | |
| (USP), | | | | | | | | |
| C2H3NaO2 | | | | | | | | |
| Magnesium | 214.41 | | | | | | | 0.0778 |
| Citrate, | | | | | | | | |
| C6H6MgO7 | | | | | | | | |
| Magnesium | 451.12 | | | | | | | |
| Citrate, | | | | | | | | |
| Anhydrous | | | | | | | | |
| (USP), | | | | | | | | |
| C12H10Mg3 | | | | | | | | |
| O14 | | | | | | | | |
| NaCl | 58.44 | | | 0.891 | 0.406 | 0.868 | | |

TABLE 19C

| | | Mass for 500 mL WB Collection (g) | | | | | | |
|---|---|---|---|---|---|---|---|---|
| Chemical | MW | CPD | CPDA-1 | AS-1 | AS-3 | AS-5 | AS-7 | APEX |
| Adenine | 135.13 | | 0.0193 | 0.03 | 0.033 | 0.033 | 0.03 | 0.0328 |
| (USP), | | | | | | | | |
| C5H5N5 | | | | | | | | |
| Citric Acid | 192.12 | 0.209 | 0.209 | | 0.0422 | | | 0.209 |
| Monohydrate | | | | | | | | |
| (USP), | | | | | | | | |
| C6H8O7 | | | | | | | | |
| Sodium | 119.98 | 0.135 | 0.135 | | 0.264 | | | 0.134 |
| Dihydrogen | | | | | | | | |
| Phosphate | | | | | | | | |
| Monohydrate | | | | | | | | |
| (USP), | | | | | | | | |
| NaH2PO4 | | | | | | | | |
| Tri-Sodium | 258.07 | 1.62 | 1.62 | | 0.568 | | | 1.61 |
| Citrate | | | | | | | | |
| Dihydrate | | | | | | | | |
| (USP), | | | | | | | | |
| C6H5Na3O7 | | | | | | | | |
| D(+)-Glucose | 180.16 | 1.62 | 2.03 | 2.2 | 1.1 | 0.901 | 1.58 | 2.03 |
| Monohydrate | | | | | | | | |
| (USP), | | | | | | | | |
| C6H12O6 | | | | | | | | |
| D(-)- | 182.17 | | | 0.825 | | 0.578 | 1.1 | 1 |
| Mannitol | | | | | | | | |
| (USP), | | | | | | | | |
| C6H14O6 | | | | | | | | |
| Di-Sodium | 141.96 | | | | | | 0.187 | 0.17 |
| Hydrogen | | | | | | | | |
| Phosphate | | | | | | | | |
| Anhydrous | | | | | | | | |
| (USP), | | | | | | | | |
| Na2HPO4 | | | | | | | | |
| Sodium | 84.01 | | | | | | 0.24 | 0.336 |
| Hydrogen | | | | | | | | |
| Carbonate | | | | | | | | |
| (USP), | | | | | | | | |
| NaHCO3 | | | | | | | | |
| Sodium | 82.03 | | | | | | | 0.41 |
| Acetate, | | | | | | | | |
| Anhydrous | | | | | | | | |
| (USP), | | | | | | | | |
| C2H3NaO2 | | | | | | | | |
| Magnesium | 214.41 | | | | | | | 0.0864 |
| Citrate, | | | | | | | | |
| C6H6MgO7 | | | | | | | | |
| Magnesium | 451.12 | | | | | | | |
| Citrate, | | | | | | | | |
| Anhydrous | | | | | | | | |
| (USP), | | | | | | | | |
| C12H10Mg3 | | | | | | | | |
| O14 | | | | | | | | |
| NaCl | 58.44 | | | 0.99 | 0.451 | 0.965 | | |

It shall be understood, of course, that the ingredients (and the amounts thereof) listed in Tables 19A-19C are simply exemplary, and should not be construed as limiting the dry red blood cell additive formulations described herein. For example, the amount of any of the ingredients may vary by + or −(i.e., +/−) 30%, or +/−25%, or +/−20%, or +/−15%, or +/−10%, or +/−5%, or +/−1%.

In further aspects, the invention provides a platelet additive dry formulation. Tables 20A and 20B provides the ingredients in weight (Table 20A) and by moles (Table 20B) in 100 mL liquid platelet additive solution.

TABLE 20A

| | | Milligrams of Ingredient in 100 mL of | |
|---|---|---|---|
| | | Platelet Additive Solution | |
| Ingredient | | (total 100 mL in sterile water) | |
| | Molecular Weight | PAS-C (Intersol) | PAS-F (Isoplate) |
| Chemical Name | (g/mol) | Liquid Solution | Liquid Solution |
| Sodium Chloride | 58.44 (anhydrous) | 452.00 | 530.00 |
| Sodium Citrate | 294.1 (anhydrous) | 279.04 | |
| Sodium Gluconate | 218.14 (anhydrous) | | 500.00 |
| Sodium Acetate | 82.03 (anhydrous) | 266.44 | 223.04 |
| Dibasic Sodium Phosphate | 141.96 (anhydrous) | 305.00 | 7.89 |
| Monobasic Sodium | 119.98 (anhydrous) | 80.86 | |
| Phosphate | | | |
| Monobasic Potassium | 136.09 (anhydrous) | | 0.82 |
| Phosphate | | | |
| Potassium Chloride | 74.55 (anhydrous) | | 37 |
| Magnesium Chloride | 95.21 (anhydrous) | | 14.05 |

TABLE 20B

| | | Millimoles of Ingredient in 100 mL of | |
|---|---|---|---|
| | | Platelet Additive Solution | |
| Ingredient | | (total 100 mL in sterile water) | |
| | Molecular Weight | PAS-C (Intersol) | PAS-F (Isoplate) |
| Chemical Name | (g/mol) | Liquid Solution | Liquid Solution |
| Sodium Chloride | 58.44 (anhydrous) | 7.7344 | 9.0691 |
| Sodium Citrate | 294.1 (anhydrous) | 1.0813 | |
| Sodium Gluconate | 218.14 (anhydrous) | | 2.291 |
| Sodium Acetate | 82.03 (anhydrous) | 3.2481 | 2.719 |
| Dibasic Sodium Phosphate | 141.96 (anhydrous) | 2.1485 | 0.0555 |
| Monobasic Sodium | 119.98 (anhydrous) | 0.674 | |
| Phosphate | | | |
| Monobasic Potassium | 136.09 (anhydrous) | | 0.0060 |
| Phosphate | | | |
| Potassium Chloride | 74.55 (anhydrous) | | 0.4963 |
| Magnesium Chloride | 95.21 (anhydrous) | | 0.1476 |

In further embodiments, Tables 21, 22, and 23 provide non-limiting examples of the lower limit (Table 21) and upper limit (Table 22), and the mean (Table 23) of chemical components in mass of a dry formulation (i.e., lacking free water molecules) of a platelet additive solution for a one unit of platelets, where each ingredient is, for example, within ±5% of the stated value.

TABLE 21
Lower Limit of Ingredients of Dry Platelet Additive Solutions
(mg per one platelet unit from one donated whole blood unit)

| Ingredient (anhydrous) | PAS-C (Intersol) | PAS-F (Isoplate) |
|---|---|---|
| Sodium Chloride | 335.77 | 393.71 |
| Sodium Citrate | 207.29 | |
| Sodium Gluconate | | 371/43 |
| Sodium Acetate | 197.93 | 165.69 |
| Dibasic Sodium Phosphate | 226.57 | 5.86 |
| Monobasic Sodium | 60.07 | |
| Phosphate | | |
| Monobasic Potassium | | 0.61 |
| Phosphate | | |
| Potassium Chloride | | 27.49 |
| Magnesium Chloride | | 10.44 |

TABLE 22
Upper Limit of Ingredients of Dry Platelet Additive Solutions
(mg per one platelet unit from one donated whole blood unit)

| Ingredient | PAS-C (Intersol) | PAS-F (Isoplate) |
|---|---|---|
| Sodium Chloride | 587.60 | 689.00 |
| Sodium Citrate | 362.75 | |
| Sodium Gluconate | | 650.00 |
| Sodium Acetate | 346.37 | 299.95 |
| Dibasic Sodium Phosphate | 396.50 | 10.25 |
| Monobasic Sodium | 105.12 | |
| Phosphate | | |
| Monobasic Potassium | | 1.07 |
| Phosphate | | |
| Potassium Chloride | | 48.10 |
| Magnesium Chloride | | 18.26 |

TABLE 23
Mean weight of Ingredients of Dry Platelet Additive Solutions
(mg per one platelet unit from one donated whole blood unit)

| Ingredient | PAS-C (Intersol) | PAS-F (Isoplate) |
|---|---|---|
| Sodium Chloride | 461.69 | 541.36 |
| Sodium Citrate | 285.02 | |
| Sodium Gluconate | | 510.71 |
| Sodium Acetate | 272.15 | 227/92 |
| Dibasic Sodium Phosphate | 311.54 | 8.05 |
| Monobasic Sodium | 82.59 | |
| Phosphate | | |
| Monobasic Potassium | | 0.84 |
| Phosphate | | |
| Potassium Chloride | | 37.79 |
| Magnesium Chloride | | 14.35 |

It shall be understood, of course, that the ingredients (and the amounts thereof) listed in Tables 21-23 are simply exemplary, and should not be construed as limiting the dry platelet additive formulations described herein. For example, the amount of any of the ingredients may vary by + or −(i.e., +/−) 30%, or +/−25%, or +/−20%, or +/−15%, or +/−10%, or +/−5%, or +/−1%.

It will be understood that the dry formulations provided herein are examples for dry red blood cell additives, dry platelet additives, and dry anticoagulant/preservative formulations for whole blood based on the liquid formulations currently used. But as the solid formulations of the invention do not add any free water, certain salts (e.g., sodium chloride and potassium chloride) that are included in liquid formulation to maintain osmotic pressure across cell membranes may be reduced by as much as 65% in the dry formulations described herein. In some embodiments, the red blood cell fraction separated from the whole blood is suspended in plasma that has not been diluted by any free water present in the anticoagulant-preservative formulation added to the whole blood from which the red blood cell fraction was separated. In some embodiments, the platelet fraction separated from the whole blood in suspended in plasma that has not been diluted by any free water present in the anticoagulant-preservative formulation added to the whole blood from which the plasma fraction was separated. In some embodiments, the plasma is separated from the whole blood has not been diluted by any free water present in the anticoagulant-preservative formulation added to the whole blood from which the plasma was separated.

Table 24 below lists non-limiting examples of potential ingredients of an anticoagulant/preservative formulations for storage of whole blood (citrate+other constituents per mL WB Collection) that can be included in the dry formulations described herein. Note that the target concentration, minimal concentration and maximum target concentration in Table 24 are merely examples, and should not be considered limiting in any way. For example, the amount of any of the ingredients may vary by + or −(i.e., +/−) 30%, or +/−25%, or +/−20%, or +/−15%, or +/−10%, or +/−5%, or +/−1%.

TABLE 24
Potential ingredients of anticoagulant/preservative formulations for storage
of Whole Blood (citrate + other potential constituents per mL WB collection)

| | | Target | Min | Max | |
|---|---|---|---|---|---|
| Ingredient | Type | (umol) | (umol) | (umol) | Reasoning and Comments |
| Citrate | Acid | 11.76 | 5.88 | 23.52 | Anticoagulant needed for blood |
| | and/or | | | | collection and buffering. The ratio |
| | salt | | | | of citrate acid to Sodium citrate may |
| | | | | | be adjusted for the desired target |
| | | | | | pH. Min value determined based on |
| | | | | | 0.5* Target (like 0.5 CPD). |
| Phosphate | Acid | 1.80 | 0 | 6.98 | Not likely needed for RBCs as |
| | and /or | | | | phosphate is mainly provided for |
| | Salt | | | | preventing phosphate leakage from |
| | | | | | RBCs. It does improve buffering |
| | | | | | capacity and may be needed in |
| | | | | | combination with acetate for |
| | | | | | platelets. Shimizu and Murphy |
| | | | | | reported using is at 25 mM |
| | | | | | phosphate and 25 mM |
| | | | | | concentration to replace plasma. |
| | | | | | Transfusion. 1993 Apr.; 33(4): 304-10. |
| | | | | | doi: 10.1046/j.1537- |
| | | | | | 2995.1993.33493242637.x. These |
| | | | | | amounts may be excessive for |
| | | | | | transfusion. |
| Dextrose | Sugar | 22.54 | 0 | 37.86 | Dextrose is consumed and is needed |
| | | | | | to extend storage and the amount |
| | | | | | will be dependent of length of |
| | | | | | intended storage. likely values for |
| | | | | | typical storage to be in the range of |
| | | | | | that of CPD and CP2D. Value suggest |
| | | | | | is that of CPDA-1 but with better |
| | | | | | storage different amount may be |
| | | | | | chosen such as that of CPDA-2 |
| | | | | | (1.75x CPD or equivalently |
| | | | | | 1.4*CPDA-1) which would be 15.78 |
| | | | | | mmol per WB collection. suggested |
| | | | | | value chosen to maintain the |
| | | | | | osmolarity of solution low and as |
| | | | | | needed. |
| Adenine | Nucleotide | 0.285 | 0 | 0.570 | Adenine is consumed and assists |
| | | | | | with ATP maintenance. Value |
| | | | | | chosen based on CPDA-1 but other |
| | | | | | values may be chosen such as that of |
| | | | | | CPD. |
| Guanosine | Nucleoside | 0.165 | 0 | 0.66 | Guanosine is consumed and assists |
| | | | | | with 2-3 DPG maintenance. Value |
| | | | | | chosen based on PAGGS-M |
| | | | | | formulation. |
| D-Mannitol | Sugar | 11 | 0 | 22 | D-Mannitol is added as a RBC |
| | Alcohol | | | | Membrane stabilizer. Other sugar |
| | | | | | alcohols may be chosen in |
| | | | | | combination or standalone but |
| | | | | | mannitol is the primary sugar |
| | | | | | alcohol currently in use for RBC |
| | | | | | storage. Use of mannitol for WB |
| | | | | | storage may provide addition |
| | | | | | cellular membrane protection. |
| Acetate | Salt | 10 | 0 | 20 | Acetate metabolizes to carbon |
| | | | | | dioxide by the platelets. |
| Magnesium | Salt | 0.70 | 0 | 1.6 | Magnesium prevents platelet |
| | | | | | cytoskeleton storage lesions. |
| L-Carnitine | Amino | 7 | 0 | 14 | L-Carnitine and other antioxidants |
| | acid | | | | may be used in the formulation to |
| | | | | | combat the effects of storage- |
| | | | | | induced oxidative stress in platelets |
| | | | | | and RBCs. it may also act as anti- |
| | | | | | microbial (bacterial, viral, protozoal, |
| | | | | | and fungal) protection. The normal |
| | | | | | blood levels in humans are 50-60 |
| | | | | | μmol/L for total carnitine and 40-50 |
| | | | | | μmol for free carnitine. L-Carnitine is |
| | | | | | available as USP ingredient and is |
| | | | | | used for carnitine deficiency and the |
| | | | | | usual dose is 50 mg per kg (22.7 mg |
| | | | | | per pound) of body weight a day |
| | | | | | injected into a vein. The drug may |
| | | | | | interact with anticoagulant such as |
| | | | | | Warfarin and Acenocoumarol (a |
| | | | | | vitamin K antagonist). Ref: 1. |
| | | | | | Ravikumar et al, “Effects of L- |
| | | | | | carnitine on the erythrocytes of |
| | | | | | stored human blood”, 2. Transfusion |
| | | | | | Medicine 2019. Sci J Iran Blood |
| | | | | | Transfus Organ 2018; 15(1): 10-11 |
| Bicarbonate | Salt | 8 | 0 | 16 | Bicarbonate as a buffer and the |
| | | | | | suggested amount would raise initial |
| | | | | | bicarbonate content from the mean |
| | | | | | value to upper range of adults. As |
| | | | | | acetate is consumed by platelets |
| | | | | | some added buffering is provided |
| | | | | | during storage. |

In some embodiments, the anticoagulant component of the formulation such as citrate is an important component of WB storage solution as it is required to prevent blood clotting during blood collection. Other constituents in the formulation may aid in cellular membrane stability, appropriate pH maintenance and adequate buffering capacity of suspension, and nutrients such as dextrose, adenine, acetate are needed for cellular function of cells, etc.

As such, in some embodiments, non-limiting dry formulations may be prepared based on the ingredients in the above tables where the anticoagulant may be combined with one or more of the other ingredients to form a dry or solid anticoagulant/preservative formulation. The dry formulations described herein are not limited to such ingredients and may be combined with other ingredients to enhance the solubility, and release of the ingredients for blood storage. It should also be understood that the particulates in the dry formulation may be of any size. Thus, powders and mixtures of powders are included in the dry or solid formulations described herein.

It should be noted that a non-limiting formulation of whole blood (WB) anticoagulant/preservative in solid form, the same concept may be applied to process WB into blood components, such as blood components with reduced plasma including red blood cells.

In some embodiments of the invention, water or other liquids may be added to a solid formulation as described herein prior to the addition of the whole blood and/or blood component.

It will be understood that in various embodiments, any dry formulation of the invention may be in a powder form. In another embodiment, any dry formulation of the invention may be in a form of one or more tablets. In another embodiment, any dry formulation of the invention may be in a form of one or more capsules. In various embodiments, any dry formulation of the invention may be in the form of a combination of different solid forms. For example, a single dry formulation of the invention may comprise dextrose in powder and sodium citrate and citric acid combined in a single tablet. In another example, a single formulation of the invention may comprise a tablet of dextrose, a tablet of citric acid, and sodium citrate in powder form. In another non-limiting example, a single formulation of the invention may comprise a tablet of dextrose and a citrate-providing agent, such as sodium citrate or citric acid, in powder form.

It shall be understood that in the various embodiments of the various dry formulations of the invention, the formulations (e.g., powders, tablets, etc.) may additionally include inactive ingredients such as excipients that are used in standard tablet manufacturing. Excipients include diluents, fillers, binders, glidants, and lubricants. In some embodiment, where the formulation contains one or more tablets, the formulation additionally includes excipients that aid in the formation of the tablets.

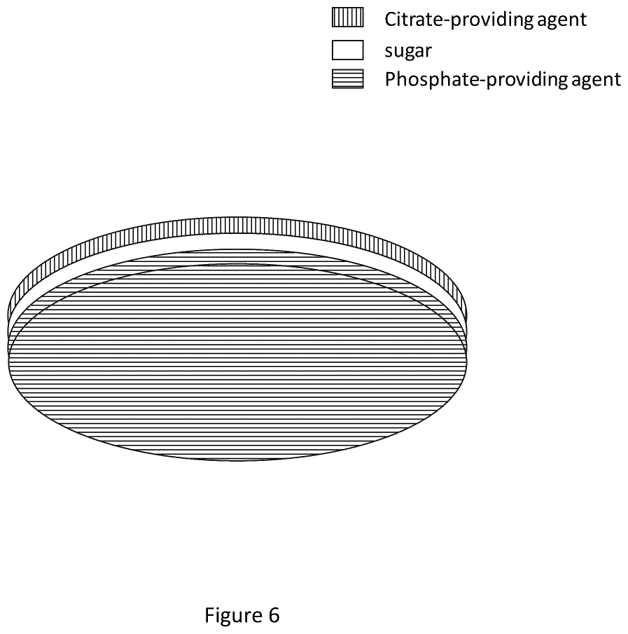

In one embodiment of solid formulation, tablet composition is designed for the ordered release of active ingredients. The said tablet composition includes a core layer and at least one additional layer, each partially or completely encapsulating distinct active ingredients. A non-limiting example of such a tablet is shown in FIG. 6.

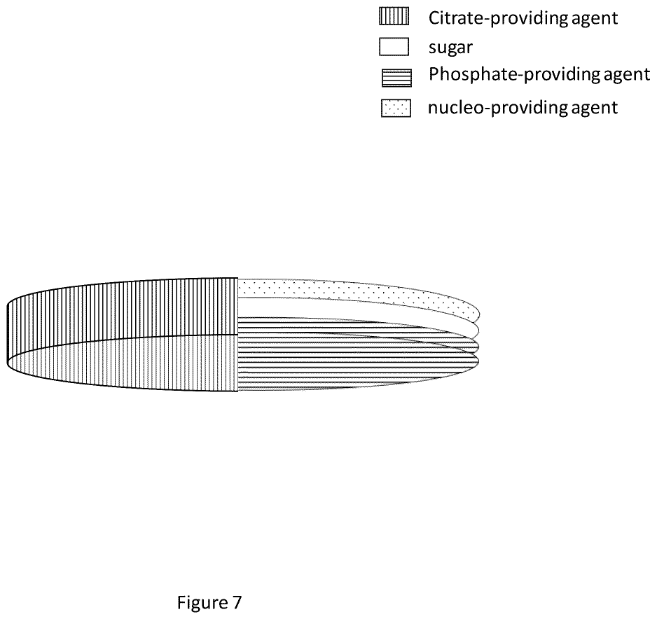

In some embodiments, the core layer of the tablet is comprised of a first active ingredient. The active ingredient may be any pharmaceutical compound, nutrient, supplement, or other chemical compound that serves a blood preservation function for blood or blood component storage when in contact with blood. In FIG. 6, the core layer of the non-limiting tablet shown in a sugar, such as dextrose. In some embodiments, a citrate-providing agent (e.g., citric acid or sodium citrate) may be included in the outer layer of a multi-layer tablet, or in a coating of a tablet), so that the citrate-providing agent is released first into the whole blood (or blood component such as red blood cells). As shown in FIG. 7, a multi-layer tablet may be coated with a citrate-providing layer. A mono-layer tablet (e.g., a tablet comprising dextrose) may also be coated with a citrate-providing layer (not shown).

In some embodiments, in a multi-layer or coated tablet, the citrate-providing agent is released into the whole blood (or blood component such as red blood cells) prior to the release of a phosphate-providing agent. In some embodiments, in a multi-layer or coated tablet, the citrate-providing agent is released into the whole blood (or blood component such as red blood cells) prior to the release of a sugar, such as dextrose. In some embodiments, in a multi-layer or coated tablet, the citrate-providing agent is released into the whole blood (or blood component such as red blood cells) prior to the release of a nucleotide (or nucleoside)-providing agent, such as adenine or guanosine.

In some embodiments, a non-limiting tablet further includes one or more additional layers, each adhered to the core layer or to another additional layer. Each of these additional layers partially or completely encapsulates a different active ingredient. The additional active ingredients may also be any pharmaceutical compounds, nutrients, supplements, or other chemical compounds.

One non-limiting distinguishing feature of a non-limiting tablet as described herein is the ordered release of the active ingredients, achieved through the particular design and formulation of the tablet layers, with or without the use of rate-controlling membranes or fillers. Each layer is designed to dissolve or disintegrate at different rates upon administration, resulting in a sequential release of the active ingredients in a predetermined order.

The rate of dissolution or disintegration and thus the release of each active ingredient is determined by the intrinsic properties of each layer, such as the density, porosity, and the solubility of the active ingredient within each layer. Factors such as the tablet's shape, size, weight, hardness, or other physical properties may also be manipulated to control the dissolution rates of the layers. That rate of release from each layer or the solid form as a whole may be characterized as this is typical in drug formulation and as a zero-order release, first order release, and Higuchi, Korsmeyer-Peppas, Hopfenberg, and Weibull models or other release models as suited for the application.

In some embodiments, the tablet composition described herein provides a method for the timed release of multiple active ingredients, improving the efficacy and the safety profile of pharmaceutical compounds, nutrients, supplements, and other chemical compounds. This invention provides a significant advancement in the fields of blood storage, blood banking and transfusion medicine.

Thus, in some embodiments, the formulations described herein may include a wide variety of pharmaceutical excipients useful for liquid protein formulations that are known to those skilled in the art. They include one or more additives, such as liquid solvents or co-solvents; sugars or sugar alcohols such as mannitol, trehalose, sucrose, sorbitol, fructose, maltose, lactose, or dextrans; surfactants such as TWEEN® 20, 60, or 80 (polysorbate 20, 60, or 80); buffering agents; preservatives such as benzalkonium chloride, benzethonium chloride, tertiary ammonium salts, and chlorhexidinediacetate; carriers such as poly(ethylene glycol) (PEG); antioxidants such as ascorbic acid, sodium metabisulfite, and methionine; chelating agents such as EDTA or citric acid; or biodegradable polymers such as water soluble polyesters; cryoprotectants; lyoprotectants; bulking agents; and stabilizing agents.

Other pharmaceutically acceptable carriers, excipients, or stabilizers, such as those described in Remington: “The Science and Practice of Pharmacy”, 20th edition, Alfonso R. Gennaro, Ed., Lippincott Williams & Wilkins (2000) may also be included in a protein formulation described herein, provided that they do not adversely affect the desired characteristics of the formulation or safety of the blood recipient patient.

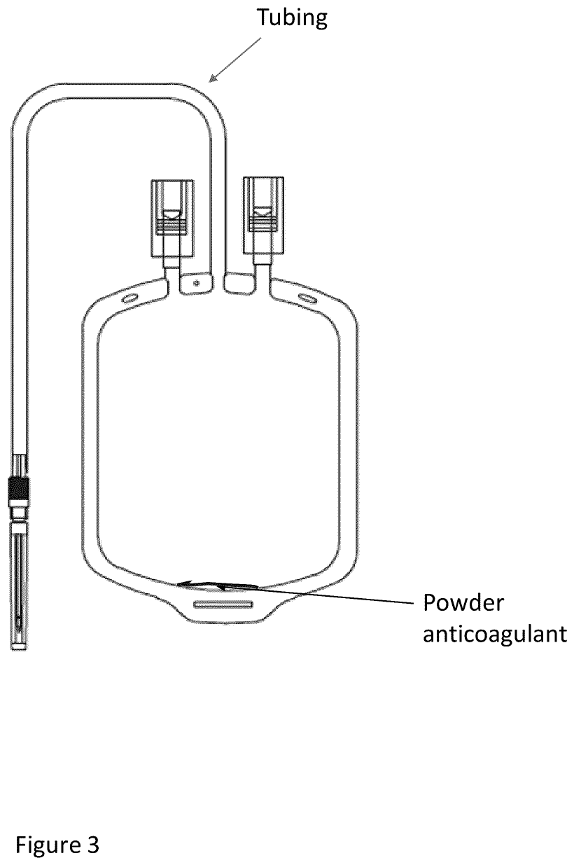

In further aspects, the invention provides a blood bag that comprises an interior, where a dry or solid formulation of the invention is contained within the interior of the bag. The formulation may be in any solid form, including, without limitation, a tablet, a powder, a capsule, a gel, or any combination of any solid form (e.g., some components in tablet form and some components in powder form). In some embodiments, the dry formulation may coat all or some of the interior surface of the blood bag. In some embodiments, the blood bag is adapted to receive whole blood for a donor (e.g., a human donor). Blood bags are well known. FIG. 3 provides a schematic where an anticoagulant/preservative formulation of the invention, in the form of powder, is within the interior of a blood bag.

In some embodiments, the solid formulation described herein may be added to the blood bag during the manufacture of the blood bag. Using the schematic shown in FIG. 3 as a non-limiting example, powder can be added to the blood bag prior to sealing the bag or port and prior to sterilization of the bag by gamma irradiation. Where the formulation is in tablet format, the tablet (or tablets if multiple tablets are combined to result in the formulation) can be added to the blood bag while the blood bag is being manufactured, prior to sealing the bag.

As mentioned, a dry or solid formulation described herein need not be contained within a single tablet. Likewise, the tablets of the formulation need not be uniform. Without wishing to be bound by a particular theory, it may be efficient to package each chemical ingredient of the formulation in separate tablets, and each tablet can then be added to or incorporated into the blood bag prior to sterilization. For example, components of the formulation that do not include an energy-providing agent (e.g., sugar) can be sterilized by dry heat.

In a non-limiting example, a standard tablet manufacturing device (e.g., the NP-RD30 multifunction rotary tablet press available from Natoli Engineering, St. Charles County, Missouri, USA) can be employed. For a set of tablets to be added to a bag into which about 500 mL donated whole blood will be added, three tablets are pressed as follows:
Tablet 1:1350 mg sodium citrate dihydrate
Tablet 2:490 mg citric acid monohydrate
Tablet 3:1508 mg dextrose monohydrate.

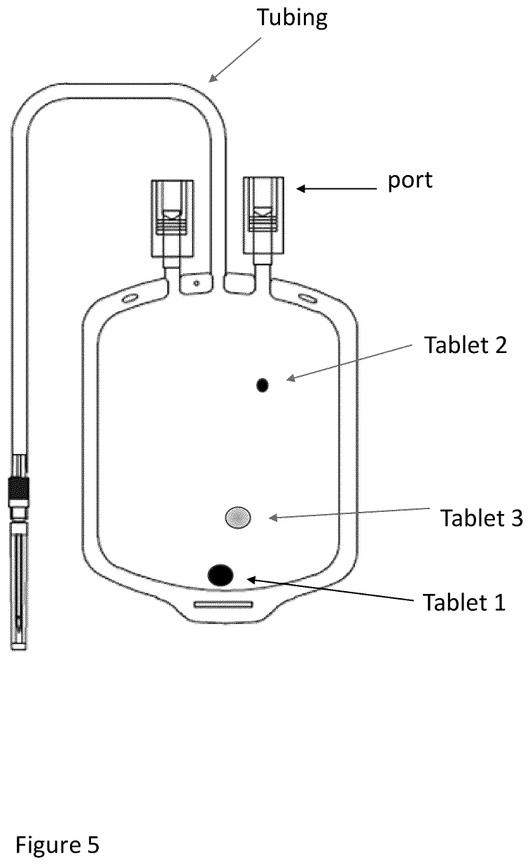

For ease of manufacture, the three tablet may be of different size or shape. For example, Tablet 2 is the smallest while Tablet 1 and Tablet 3 may have different diameters and thickness so that blood bags containing one each of Tablet 1, Tablet 2, and Tablet 3 can be readily and quickly assembled. FIG. 5 provides a schematic of such blood bag containing Tablet 1, Tablet 2, and Tablet 3 in its interior.

The rationale for solid formulations described above for anticoagulant/preservative to be mixed with whole blood is similarly applicable to additive solutions for blood components, such as red blood cell additive solutions. Thus, in some embodiments, the invention contemplates solid formulations (e.g., lacking free H2O molecules) that can be mixed with packed red blood cells.

In another aspect, the invention provides a blood bag comprising a formulation described herein. In some embodiments, the blood bag is part of a multi-blood bag set where, for example, the set contains multiple bags in-line with one another to hold blood components following processing of donated whole blood.

In some embodiments, the blood bag of the invention comprises plasticized polyvinyl chloride (PVC). In some embodiments, sheets of polymeric material comprising a non-polyvinyl chloride (PVC) including, without limitation, polyolefins, ethylene vinyl acetate (EVA), polyethylene terephthalate (PET), polyurethane (PU), Thermoplastic elastomers (TPEs), Polybutadiene or combinations of one or two of these and/or other plastics). In some embodiments, the blood bag comprises ethylene-tetrafluoroethylene (ETFE). In some embodiments, the blood bag comprises polyethylene (PE), such as high-density polyethylene. In some embodiments, the blood bag comprises polyvinylidene fluoride (PVDF). In some embodiments, the blood bag comprises Ethylene-vinyl acetate (EVA). In some embodiments, the blood bag comprises polyolefin.

Polyvinyl chloride (PVC) is a commonly used material in blood storage bags.

However, as PVC is stiff and generally heat-sensitive, a plasticizer must be added to the PVC material to give the flexibility and thermal properties needed for blood bag manufacture. A plasticizer is a substance or material incorporated in a material (e.g., PVC) to increase that material's flexibility, workability, or distensibility. Plasticizer molecules do not form chemical bonds with the polymer; rather, they are embedded in the PVC matrix, which means that plasticizer molecules can freely migrate and are often found to leach out of the bag material. The rate of leaching is different for each plasticizer and is also influenced by the handling of the bag.

Di (2-ethylhexyl) phthalate (DEHP) is a common plasticizer used in PVC bags. Typically, a PVC-DEHP bag contains 20-50% plasticizer by weight.

Accordingly, on some embodiments, the blood bag comprises PVC comprising the DEHP plasticizer.

Di(2-ethylhexyl) phthalate (DEHP) is an ortho-phthalate. The leaching of ortho-phthalates into donor blood contained within PVC bags comprising an ortho-phthalate plasticizer may result in detrimental health effects to patients receiving such donor blood.

Accordingly, in some embodiments, the blood bag described herein lack or substantially lack ortho-phthalates. For example, a blood bag that substantially lacks ortho-phthalate is 95% free, or 98% free, or 99% free, or 99.9% free, by weight, of an ortho-phthalate plasticizer such as DEHP.

In some embodiments, the blood bag comprises a non-ortho-phthalate plasticizer. In some embodiments, the non-ortho-phthalate plasticizer is n-butyryl tri-n-hexyl-citrate (BTHC). In some embodiments, the non-ortho-phthalate plasticizer is trioctyl trimellitate (TOTM). In some embodiments, the non-ortho-phthalate plasticizer is acetyl tributyl citrate (ATBC). In some embodiments, the non-ortho-phthalate plasticizer is di-(2-ethylhexyl) terephthalate (DEHT) or referred to as Di(ethylhexyl) terephthalate (DOTP). In some embodiments, the non-ortho-phthalate plasticizer is di(isononyl)cyclohexane-1,2-dicarboxylate (also called 1,2-Cyclohexane dicarboxylic acid diisononyl ester) (DINCH).

The following examples in no way limit the present invention.

### Example 1

In this example, the anticoagulant/preservative formulation CPD will be made in a dry formulation for mixing with 450 mL donated whole blood, where the dry formulation is in the form of three tablets.

For Tablet 1, 1.607 grams of Dextrose monohydrate (C6H12O₆*H2O) is formed into a tablet.

For Tablet 2, 1.657 grams of sodium citrate dihydrate (C6H5Na3O7*2H2O) is formed into a tablet.

For Tablet 3, 206 mg of citric acid monohydrate (C6H8O7*H2O) is combined with 140 mg of monobasic sodium phosphate monohydrate (NaH2PO4*H2O), and this combination is formed into a tablet.

For the tableting, a tablet press is employed. Either a single-punch or a rotary table press may be employed. For example, the XL 600 Tablet Press commercially available from Korsch America, Inc. (South Easton, Massachusetts, USA) may be employed to generate the tablets.

Packets containing one each of the three tablets are prepared for addition to whole blood.

### Example 2

This example describes the manufacture of a blood bag containing a solid formulation as described herein.

Sheets of Polyvinyl chloride (PVC) with plasticizer are used for the blood bags. In this example, the plasticizer will be 1,2-Cyclohexane dicarboxylic acid, diisononyl ester (DINCH).

The blood bags are prepared according to industry standards. Briefly, the DINCH-plasticized PVC sheets are extruded through a die to convert it to a sheet form. The extruded sheet is then cut into the size and shape of the desired blood bag (e.g., for 450 ml or 500 mL donor blood). Prior to adding the port and tubing and sealing the bag periphery, a powder formulation, as described herein, is added. For example, for CPDA-1 for mixing with 500 mL donor blood, a homogenized powder mixture of anhydrous forms of sodium citrate, citric acid, monobasic sodium phosphate, dextrose, and adenine is prepared at the following ratio: 1.49 grams sodium citrate, 201 mg citric acid, 123.4 mg monobasic sodium phosphate, 1.9378 grams dextrose, and 20.2 mg adenine. Manufacturing equipment can add exactly 3.77 grams of this homogenized powder mixture to the bag. The powder-containing blood bag is then sealed by adding tubing and ports to the bag and sealing the periphery of the bag. The bag is then sterilized using gamma irradiation according to industry standards.

### Example 3

In this example, a tablet form of CPDA is made.

The water-containing formula for CPDA is as follows:
101.9 mM Trisodium Citrate dihydrate (89.4 mM of trisodium citrate anhydrous)
17 mM of citric Acid monohydrate (15.6 mM citric acid anhydrous)
177 mM dextrose monohydrate (161 mM of Dextrose anhydrous)
18.5 mM NaH2PO4 monohydrate (16.1 mM of NaHPO4 anhydrous)
2.035 mM adenine

### Prepared in Water for Injection (i.e., Sterile Water)

70 mL of the above CPDA anticoagulant/preservative liquid formulation is added to 500 ml+/−50 ml of whole blood collected from a donor.

If 500 mL whole blood is collected, the red blood cell and plasma volumes of whole blood (WB) at mean hematocrit (HCT) of 44% for adults 19-49 years of age is about 220 mL, and about 280 mL, respectively. To be clear, from 500 mL of collected whole blood from an adult donor, the volume of red blood cells in that 500 ml is about 220 mL, and the volume of plasma in that 500 mL is about 280 mL. The anticoagulant to plasma ratio is about 1:4 by volume (standard 500 ml collection uses 70 mLs of liquid anticoagulant/preservative formulation and as the mean volume of plasma is 280, the ratio is calculated as 70:280 or 1:4).

The total citrate concentration in the above liquid formulation is 105 mM (i.e., 89.4 mM from trisodium citrate anhydrous +15.6 mM from citric acid anhydrous). Therefore, 105 mM of citrate used at 70 mL for 500 mL of WB in which the anticoagulant to plasma ratio is about 1:4 suggests that to anticoagulate 500 ml of whole blood with 280 ml of plasma, 105 millimoles of citrate is needed to maintain the same buffering capacity. If trisodium citrate anhydrous, with a molecular weight of 258 g/moles, is solely used, about 1.8964 g of trisodium citrate anhydrous (2.1614 g trisodium dihydrate) is needed.

But free calcium ion present in plasma is about 1.1-1.4 mM (4.5-5.6 mg/dl) and in extracorporeal circuit, citrate concentrations of 15-24 mM reduce ionized Ca2+ levels sufficiently (to 0.2-0.3 mmol/L) to impair hemostasis and produce an anticoagulant effect (Ref: Lee, Grace et al., “Anticoagulation Techniques in Apheresis: From Heparin to Citrate and Beyond”, Clin Apher. 2012; 27 (3): 117-125). To achieve 21 mM of Anticoagulated WB (which is the mean citate level of WB anticoagulated, calculated as 105 mM citrate multiplied by 1/(1+4)), with CPD, CP2D, CPDA-1, or CPDA-2, 1129 mg of citrate expressed as citric acid is needed. If only trisodium citrate anhydrous is added, about 1517 mg of this ingredient is needed in the formulation to produce an anticoagulant effect. Alternatively, about 1729 mg trisodium citrate dihydrate may be used. Also, a combination of citric acid and trisodium citrate may be used.

As glucose is metabolized to lactate during storage, about the same amount of dextrose is needed in the invention. As 70 mL of CPDA-1 for 500 mL WB collection has a mean content of 31.9 g/L, 2.233 g of dextrose monohydrate or 2.030 g of dextrose anhydrous is, in some embodiments, needed for this non-limiting dry formulation.

The adenine content may be maintained the same as CPDA-1 at 19.25 mg (2.035 mM adenine at MW 135.12985=19.25 mg of adenine in 70 mL of CPDA-1).

Sodium Bicarbonate, 26 mM at a MW=84.007 g/mol, was added to make AS-7 red blood cell additive solution to replace bicarbonate that was lost when conventional processing removed plasma in making components and replaced with a bicarbonate free additive solution.

In regard to phosphate content for the formulation, one can add the same amount of phosphate to formulation as CPDA-1 but it appears that the phosphate addition was introduced to maintain the phosphate concentration of anticoagulated whole blood (“WB”) at the same level as WB that is not mixed with an anticoagulant/preservative. Therefore, in some embodiments, the dry formulation lacks phosphate. But if added, the amount of phosphate added is, in some embodiments, less than about 135 mg (1.126 moles).

Therefore, in one non-limiting embodiment, the dry or solid formulation of this non-limiting example comprises about:
1129 mg of total Citrate expressed as citric acid (5.88 mmol),
2.030 g of Dextrose (11.27 mmol), and
1.925 mg of adenine (0.1425 mmol).

It should be noted that 5.88 mmol citrate can be achieved by the inclusion of, for example, 1517 mg of trisodium Citrate anhydrous (5.88 mmol), 1729 mg of trisodium citrate dihydrate (5.88 mmol), 1129 mg of total citric acid (5.88 mmol), or any combination of trisodium citrate anhydrous, trisodium citrate dihydrate, or citric acid to arrive at 5.88 mmol citrate.

The described embodiments of the invention are intended to be merely exemplary and numerous variations and modifications will be apparent to those skilled in the art. All such variations and modifications are intended to be within the scope of the present invention.

1. A dry formulation lacking free H₂O molecules, mixing of said formulation with donated whole blood or a blood product thereof creating a formulation-treated blood for transfusion into a recipient patient.

2. The formulation of claim 1, wherein the formulation is in a form selected from the group consisting of a tablet, a powder, a capsule, a gel, and a combination of the foregoing.

3. The formulation of claim 1, wherein the formulation is mixed with 450 mL (+/−10%) of donated whole blood.

4. The formulation of claim 1, wherein the formulation is mixed with 500 mL (+/−10%) of donated whole blood.

5. The formulation of claim 1, wherein the formulation holds the formulation-treated blood at a pH of between about 6.8 to about 7.6.

6-7. (canceled)

8. The formulation of claim 1, comprising an anticoagulant component and an energy-providing component.

9. The formulation of claim 8, comprising an anticoagulant component and a preservative component.

10. The formulation of claim 8, wherein the anticoagulant component comprises citrate.

11. The formulation of claim 8, wherein the energy-providing component is dextrose.

12. The formulation of claim 8, further comprising a buffering component.

13. The formulation of claim 12, wherein the buffering component is selected from the group consisting of phosphate, bicarbonate, and citrate.

14. The formulation of claim 1, wherein the formulation-treated blood is stored for at least two days prior to transfusion into the recipient patient.

15-16. (canceled)

17. The formulation of claim 1, wherein the formulation-treated blood is stored for a period of time selected from the group consisting of at least twenty-eight days, at least thirty-five days, at least forty-two days, at least forty-nine days, and at least fifty-six days, prior to transfusion into the recipient patient.

18-20. (canceled)

21. A blood bag comprising an interior, said interior containing the formulation of claim 1.

22. The blood bag of claim 21, wherein the bag is sterilized by a sterilization method that is not steam sterilization.

23. (canceled)

24. The blood bag of claim 22, wherein the sterilization method is selected from the group consisting of gamma irradiation sterilization, x-ray sterilization, and e-beam sterilization.

25. Formulation-treated blood created by mixing donated whole blood or a blood product thereof with the formulation of claim 1.

26. The formulation-treated blood of claim 25, wherein the formulation treated blood is whole blood.

27. (canceled)

28. The formulation-treated blood of claim 25, wherein the formulation-treated blood is red blood cells.

29. (canceled)

30. The formulation-treated blood of claim 25, wherein the formulation-treated blood is leukoreduced.
